(19) United States
(12) Patent Application Publication
(10) Pub. No.: US 2024/0082529 A1
(43) Pub. Date: Mar. 14, 2024

(54) HEAT AND MOISTURE EXCHANGERS

(71) Applicant: ResMed Pty Ltd, Bella Vista (AU)

(72) Inventor: Muditha Pradeep DANTANARAYANA, Sydney (AU)

(21) Appl. No.: 18/463,948

(22) Filed: Sep. 8, 2023

(30) Foreign Application Priority Data
Sep. 9, 2022 (AU) 2022902604

(51) Int. Cl. A61M 16/10; A61M 16/06

(52) U.S. Cl. CPC A61M 16/1045; A61M 16/06

(57) ABSTRACT

Heat moisture exchanger (HME) constructions are provided for use within respiratory therapy systems. In examples, the HME constructions provide a plurality of airflow channels with different orientations to allow for improved diffusion, and/or CO2 washout within patient interfaces.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hypoventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 1.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

### 1.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

A range of patient interface seal-forming structure technologies are disclosed in the following patent applications, assigned to ResMed Limited: WO 1998/004310; WO 2006/074513; WO 2010/135785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO 2004/073778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063328 and WO 2006/130903 (describing amongst other things aspects of the ResMed Limited MIRAGE LIBERTY™ full-face mask); International Patent Application WO 2009/052560 (describing amongst other things aspects of the ResMed Limited SWIFT™ FX nasal pillows).

### 1.2.3.1.2 Positioning and Stabilising Structure

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face. Several factors may be considered when comparing different positioning and stabilising techniques. These include: how effective the technique is at maintaining the seal-forming structure in the desired position and in sealed engagement with the face during use of the patient interface; how comfortable the interface is for the patient; whether the patient feels intrusiveness and/or claustrophobia when wearing the patient interface; and aesthetic appeal.

### 1.2.3.1.3 Pressurised Air Conduit

In one type of treatment system, a flow of pressurised air is provided to a patient interface through a conduit in an air circuit that fluidly connects to the patient interface at a location that is in front of the patient's face when the patient interface is positioned on the patient's face during use. The conduit may extend from the patient interface forwards away from the patient's face.

### 1.2.3.1.4 Pressurised Air Conduit Used for Positioning/Stabilising the Seal-Forming Structure

Another type of treatment system comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy (RPT) device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

It is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

One known RPT device used for treating sleep disordered breathing is the S9 Sleep Therapy System, manufactured by ResMed Limited. Another example of an RPT device is a ventilator. Ventilators such as the ResMed Stellar™ Series of Adult and Paediatric Ventilators may provide support for invasive and non-invasive non-dependent ventilation for a range of patients for treating a number of conditions such as but not limited to NMD, OHS and COPD.

The ResMed Elisée™ 150 ventilator and ResMed VS III™ ventilator may provide support for invasive and non-invasive dependent ventilation suitable for adult or paediatric patients for treating a number of conditions. These ventilators provide volumetric and barometric ventilation modes with a single or double limb circuit. RPT devices typically comprise a pressure generator, such as a motor-driven blower or a compressed gas reservoir, and are configured to supply a flow of air to the airway of a patient. In some cases, the flow of air may be supplied to the airway of the patient at positive pressure. The outlet of the RPT device is connected via an air circuit to a patient interface such as those described above.

### 1.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 1.2.3.5 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focussed airflow.

ResMed Limited has developed a number of improved mask vent technologies. See International Patent Application Publication No. WO 1998/034665; International Patent Application Publication No. WO 2000/078381; U.S. Pat. No. 6,581,594; US Patent Application Publication No. US 2009/0050156; US Patent Application Publication No. 2009/0044808.

### 1.2.4 Heat and Moisture Exchanger (HME)

HMEs may be utilized in RPT therapy, such as in PAP therapy, to partially recover heat and moisture present in exhaled gas from a patient's airways. This heat and moisture can be retained and recycled to the patient in a passive manner as a flow of breathable gas passes through the HME prior to inspiration. Thus, the use of HMEs can provide the needed moisture and humidity (generally recognized as >10 mg/l) to most patients during PAP therapy to minimize any detrimental effects associated with PAP therapy with non-humidified ambient air whilst avoiding the need for a heated humidifier system. The use of a HME rather than a heated humidifier may also lower the possibility of occlusion caused by condensation in air delivery tubes.

The use of a HME in PAP therapy can avoid the need for additional power required with heated humidifiers and may reduce the need for extra associated components. This may reduce the manufacturing costs and also reduce the overall size of the CPAP therapy unit.

Heat and moisture exchangers are generally made up of an exchanging material, such as foam, paper, or a substance capable of acting as a condensation and absorption surface. The material may carry hygroscopic salts to improve the water-retaining capacity. Suitable salts include calcium chloride.

A problem common with the use of HMEs in CPAP therapy relates to the ability of the HME to provide sufficient heat and moisture while also minimizing flow impedance and maintaining comfortable and safe levels of CO2 washout, i.e. removing CO2 from the patient interface. Flow impedance may affect patient breathing effort (work of breathing) and also impacts event (apnoea, hypopnoea, snore) detection algorithms so in many cases it is sought to be minimized. Furthermore, consideration should also be given to heat and moisture loss from venting to ensure that the HME is functioning to counteract this loss.

It can be advantageous to design HMEs for use in RPT therapy which provide adequate patient humidification, while minimising issues with flow impedance and/or CO2 washout. For example, placing the HME unit within the elbow, around the exhaust vent or on the flow generator side of the therapy system may result in issues with impedance, and/or CO2 washout with negligible patient humidification (hygroscopic) benefit. In this configuration the vent flow may be the dominant flow through the HME, the vent flow being the flow from the patient or the flow generator that flows through the HME and directly out through the vent. Moreover, some current designs of HMEs do not allow for sufficient moisture exchange during patient exhalation to provide sufficient humidification levels to the patient. Thus, there is a need to provide superior configurations and designs for HME use in RPT therapy, such as PAP therapy, to achieve desired patient humidification whilst having acceptable impedance on the flow of therapy and CO2 washout.

It is an object of the present invention to address at least one of the foregoing problems or at least to provide the public with a useful choice.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a positioning and stabilising structure configured to provide a force to hold the seal-forming structure in a therapeutically effective position on the patient's head. The positioning and stabilising structure includes at least one strap.

One form of the present technology comprises a patient interface comprising a plenum chamber, a seal-forming structure, and a positioning and stabilising structure.

One form of the present technology comprises a patient interface comprising a plenum chamber pressurisable to a therapeutic pressure of at least 4 cmH2O above ambient air pressure. The plenum chamber includes at least one plenum chamber inlet port sized and structured to receive a flow of air at the therapeutic pressure for breathing by a patient. The patient interface also comprises a seal-forming structure that is constructed and arranged to form a seal with a region of the patient's face surrounding an entrance to the patient's airways. The seal-forming structure has a hole therein such that the flow of air at said therapeutic pressure is delivered to at least an entrance to the patient's nares. The seal-forming structure is constructed and arranged to maintain said therapeutic pressure in the plenum chamber throughout the patient's respiratory cycle in use. The patient interface may also comprise a positioning and stabilising structure to provide a force to hold the seal-forming structure in a therapeutically effective position on the patient's head.

Another aspect of one form of the present technology is a series of modular elements that may be interconnected in order to form different styles of patient interfaces.

In one form, there are at least two versions or styles of each modular element. The versions or styles may be interchangeably used with one another in order to form different modular assemblies.

One form of the present technology comprises a diffusing heat moisture exchanger.

Another form of the technology comprises a patient interface comprising a diffusing heat moisture exchanger.

Another form of the present technology comprises a heat moisture exchanger comprising a plurality of airflow channels through which a flow of breathable gas passes in use, wherein at least one of the airflow channels is provided in a different orientation from at least one other airflow channel.

Another form of the present technology comprises a heat moisture exchanger comprising a plurality of airflow channels through which a flow of breathable gas passes in use, wherein the plurality of airflow channels comprises a first set of airflow channels and a second set of airflow channels, wherein the first set of airflow channels are provided in a first orientation and the second set of airflow channels are provided in a second orientation, wherein the first orientation is different from the second orientation.

Another aspect of one form of the present technology is a heat moisture exchanger comprising a plurality of layers, wherein each of the plurality of layers comprise a plurality of channels, and at least one of the layers comprises channels oriented at a first angle with respect to a longitudinal axis of the heat moisture exchanger, and at least one of the layers comprises channels oriented at a second angle with respect to the longitudinal axis of the heat moisture exchanger, wherein the first angle is different to the second angle.

Another aspect of one form of the present technology is a patient interface for providing respiratory therapy treatments, the patient interface comprising a heat moisture exchanger, wherein the heat moisture exchanger is positioned in the flow path of a supply of breathable gas used in the respiratory therapy treatment, wherein the flow path is between an air inlet and the airways of the patient in use, wherein the heat moisture exchanger is configured to divert at least part of the flow of breathable gas away from the flow path.

Another aspect of one form of the present technology is a respiratory therapy system comprising a RPT device configured in use to create a flow of breathable gas pressurised above ambient, a patient interface configured to deliver the flow of breathable gas to the airways of a patient, and an air delivery circuit configured to transfer the flow of breathable gas from the RPT device to the patient interface. The system may further comprise a heat moisture exchanger configured to exchange heat and/or moisture in the breathable gas inspired from the patient with heat and/or moisture in the expired gas from the patient, wherein the heat moisture exchanger is provided with a plurality of channels through which the breathable gas flows in use, and wherein at least one of the plurality of channels is provided at a different angle to one or more of the other channels.

Another aspect of one form of the technology is a heat moisture exchanger for use in a respiratory therapy system. The heat moisture exchanger may comprise a frame and an exchanging material situated at least partially within the frame. The exchanging material may comprise a plurality of channels through which a flow of breathable gas passes in use, and wherein at least one of the channels is provided in a different orientation to at least one other channel.

In certain forms, the plurality of channels may comprise a first set of airflow channels and a second set of airflow channels. The first set of airflow channels may be provided in a first orientation and the second set of airflow channels may be provided in a second orientation. The first orientation may be different from the second orientation.

In certain forms, the exchanging material may further comprise a plurality of layers, where each of the plurality of layers may comprise at least one channel. For example, the first set of airflow channels may be provided in a first layer of the plurality of layers. That is, the first layer may comprise at least one channel which extends in a first orientation. The second set of airflow channels may be provided in a second layer of the plurality of layers. The second layer may comprise at least one channel which extends in a second orientation, wherein the first orientation is different to the second orientation. The frame may further comprise a plurality of openings through which the flow of breathable gas flows in use. The plurality of openings may define a longitudinal axis of the heat moisture exchanger. In some examples the first orientation may be substantially parallel to the longitudinal axis of the heat moisture exchanger, or provided at an acute angle with respect to the longitudinal axis, for example at an angle of between approximately 30 degrees with respect to the longitudinal axis, and 60 degrees with respect to the longitudinal axis.

In certain forms, the exchanging material may further comprise a substance capable of acting as a condensation and absorption surface, such as a corrugated material, foam, paper, textile or a composite material. The exchanging material may further comprise hygroscopic salts such as calcium chloride.

According to another aspect of one form of the technology there is a patient interface for providing respiratory therapy treatment to a patient, comprising a heat moisture exchanger according to other aspects of the technology. The patient interface may further comprise a plenum chamber pressurisable to a therapeutic pressure of at least 4 cmH2O above ambient air pressure. The plenum chamber may include at least one plenum chamber inlet port sized and may be structured to receive a supply of breathable gas at the therapeutic pressure for breathing by a patient. The patient interface may further comprise a seal-forming structure constructed and arranged to form a seal with a region of the patient's face surrounding an entrance to the patient's airways. The seal-forming structure may have a hole therein such that the supply of breathable gas is delivered to at least an entrance to the patient's nares. The seal-forming structure may be constructed and arranged to maintain said therapeutic pressure in the plenum chamber throughout the patient's respiratory cycle in use.

In certain forms, the patient interface may further comprise a positioning and stabilising structure to provide a force to hold the seal-forming structure in a therapeutically effective position on the patient's head.

According to another aspect of one form of the technology, there is provided a system for providing a patient with a respiratory therapy treatment. The system may comprise a patient interface according to another aspect of the technology. The system may further comprise a respiratory therapy apparatus configured to generate the supply of breathable gas. The system may further comprise an air delivery circuit configured to facilitate transfer of the supply of breathable gas from the respiratory therapy apparatus to the patient interface.

Portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

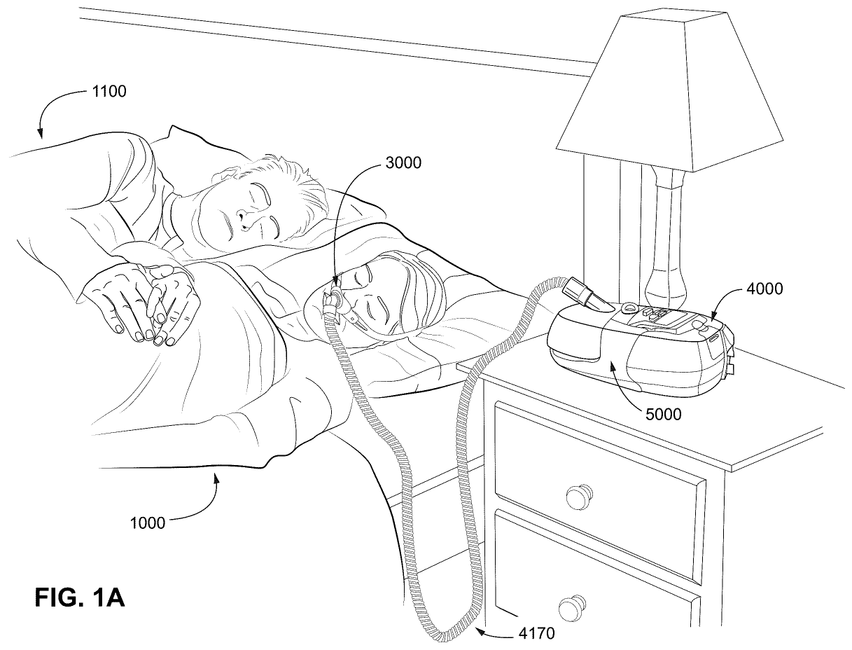

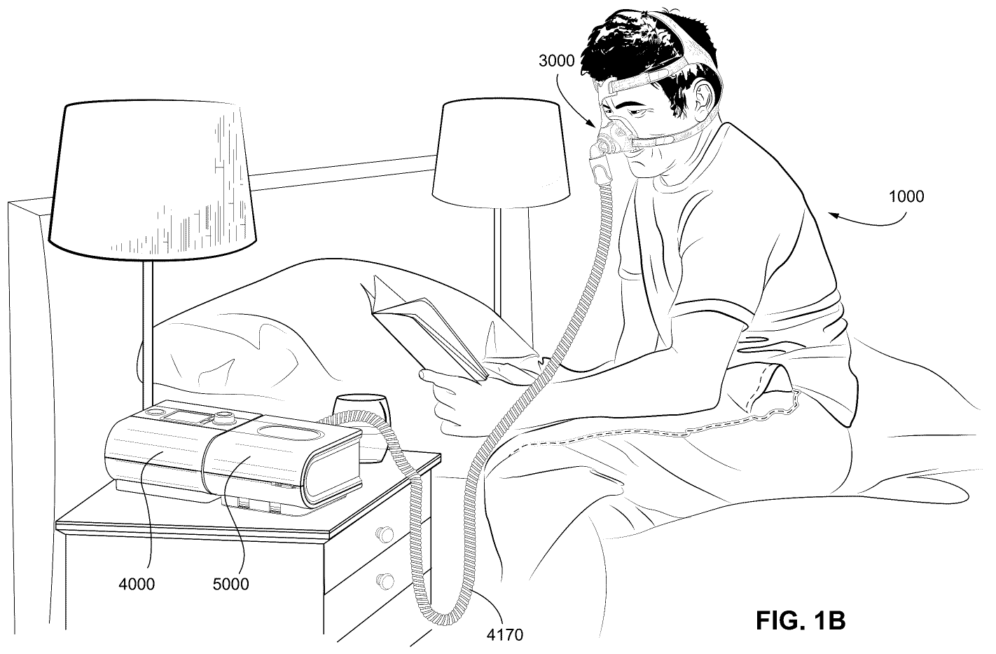

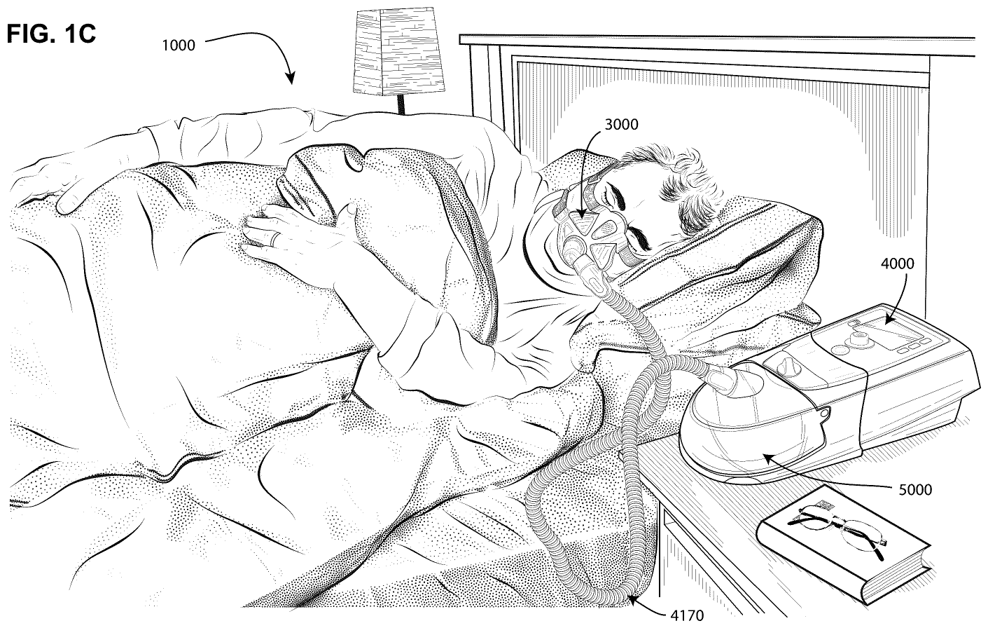

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 3.2 Respiratory System and Facial Anatomy

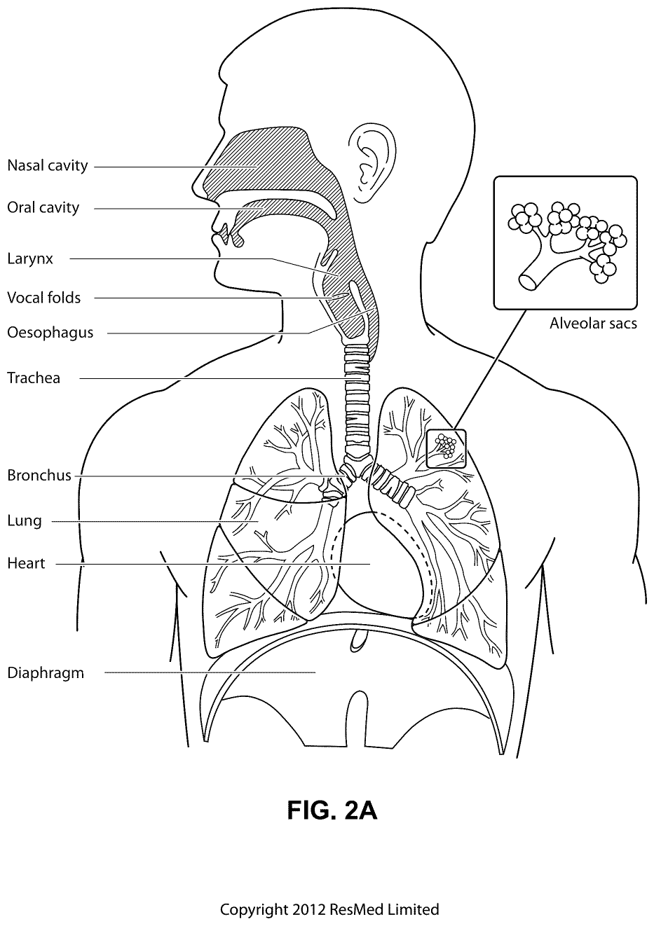

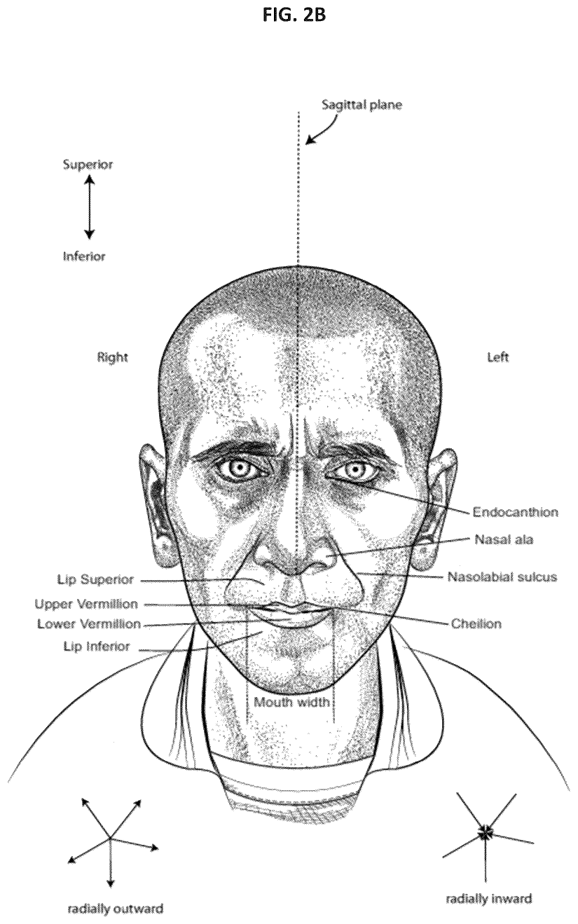

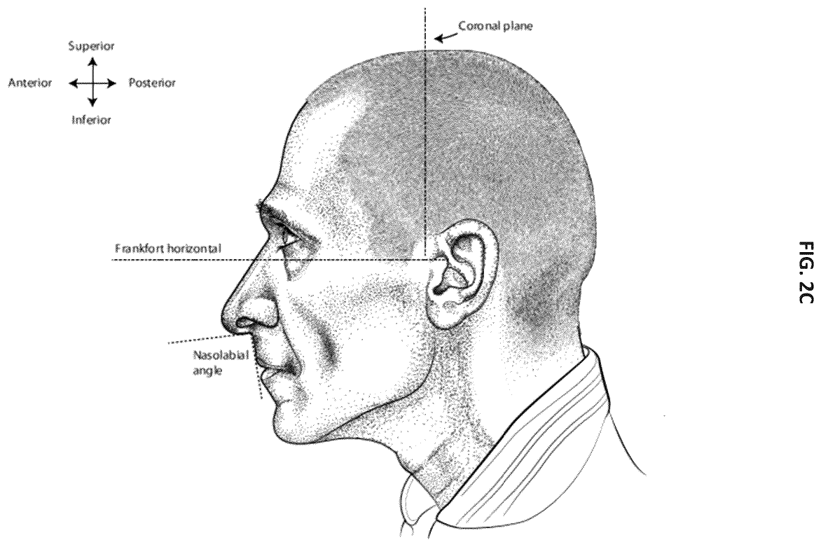

FIG. 2A shows an overview of a human respiratory system including the nasal and oral cavities, the larynx, vocal folds, oesophagus, trachea, bronchus, lung, alveolar sacs, heart and diaphragm.

FIG. 2B is a front view of a face with several features of surface anatomy identified including the lip superior, upper vermilion, lower vermilion, lip inferior, mouth width, endocanthion, a nasal ala, nasolabial sulcus and cheilion. Also indicated are the sagittal plane, directions superior, inferior, radially inward and radially outward. Also indicated are the directions superior & inferior, and anterior & posterior.

FIG. 2C is a further side view of a head. The approximate locations of the Frankfort horizontal and nasolabial angle are indicated. The coronal plane is also indicated.

### 3.3 Patient Interface

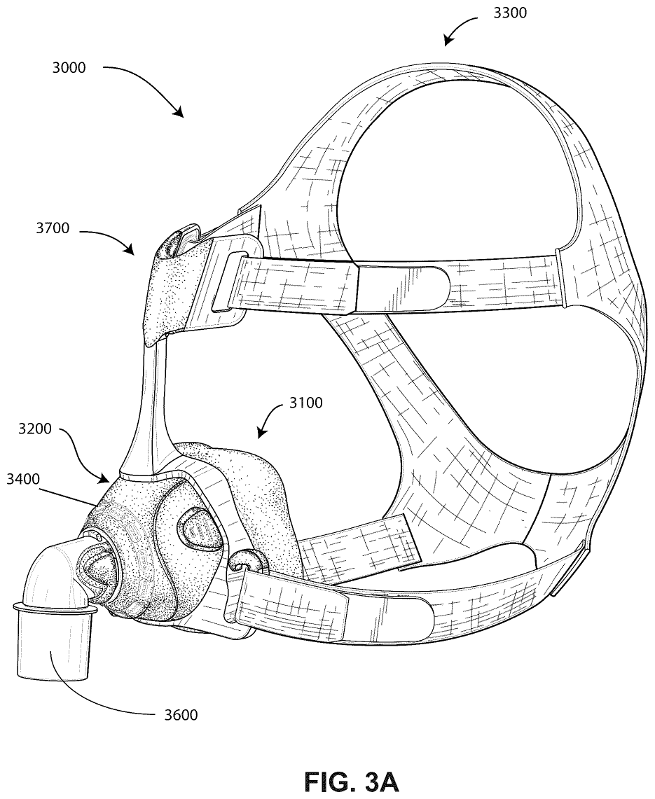

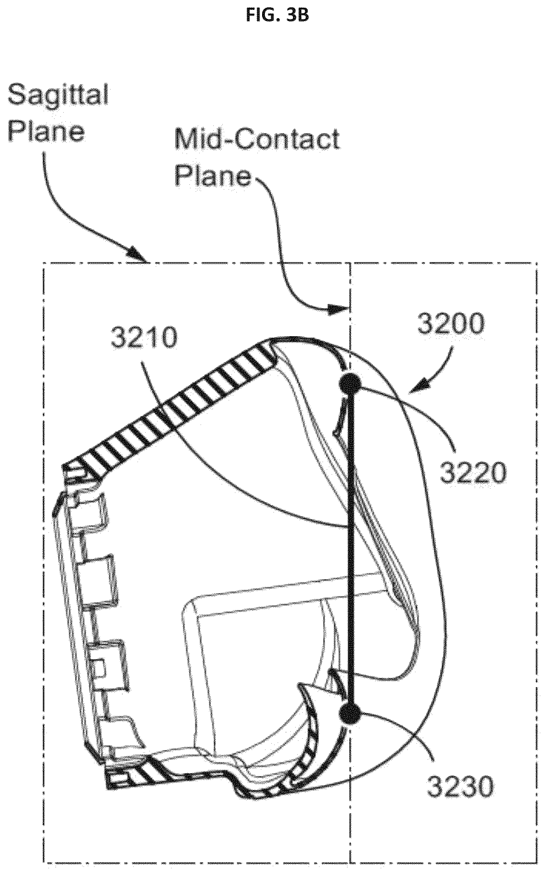

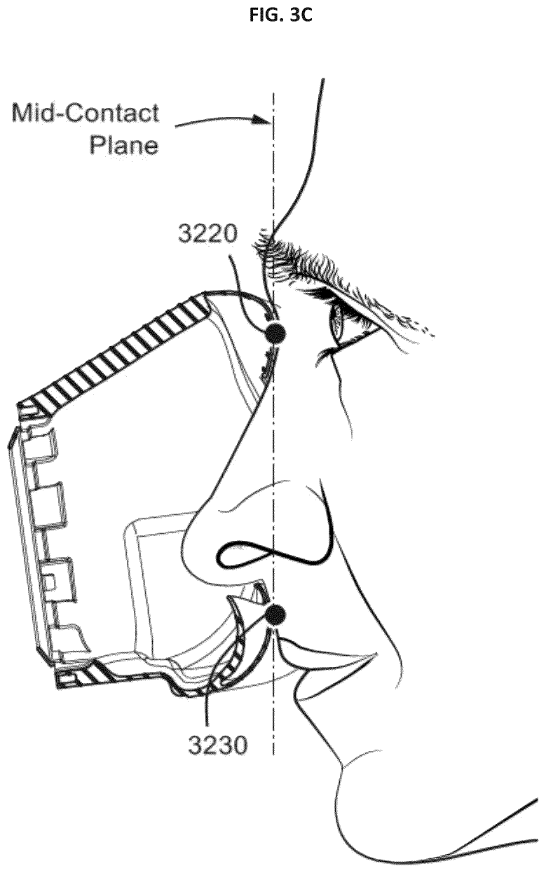

FIG. 3A shows a patient interface in the form of a nasal mask with a positioning and stabilising structure in accordance with one form of the present technology.

FIG. 3B shows a cross-section of a plenum chamber in accordance with the present technology, the cross-section being taken at the sagittal plane of the user. A ‘mid-contact’ plane is shown. The mid-contact plane is perpendicular to the sagittal plane. The orientation of the mid-contact plane corresponds to the orientation of a chord 3210 which lies on the sagittal plane and just touches the cushion of the plenum chamber at two points on the sagittal plane: a superior point 3220 and an inferior point 3230. Depending on the geometry of the cushion in this region, the mid-contact plane may be a tangent at both the superior and inferior points.

FIG. 3C shows the plenum chamber 3200 of FIG. 3B in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3C the plenum chamber 3200 is that of a nasal mask, and the superior point 3220 sits approximately on the sellion, while the inferior point 3230 sits on the lip superior.

### 3.4 Rpt Device

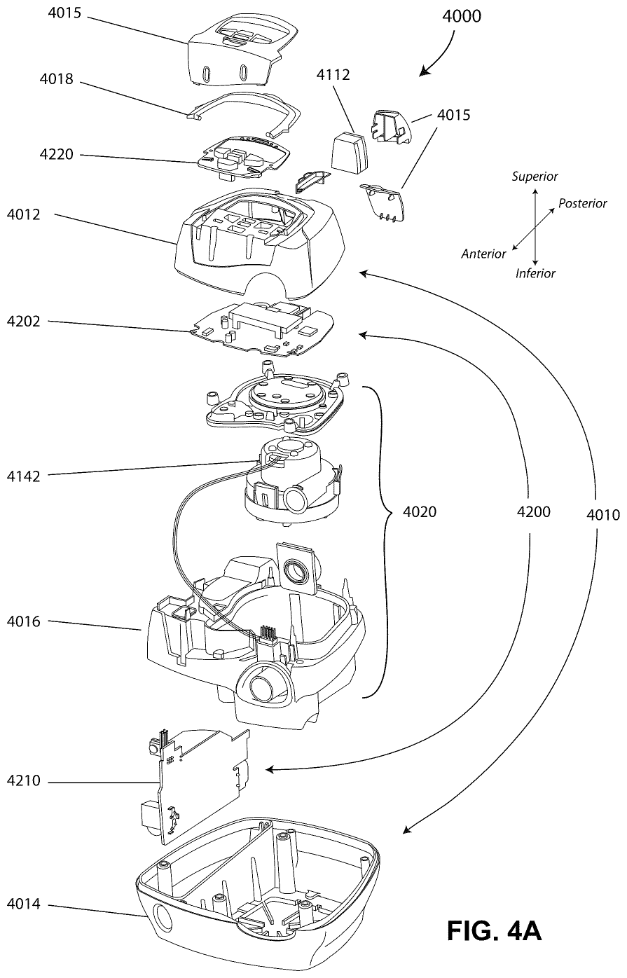

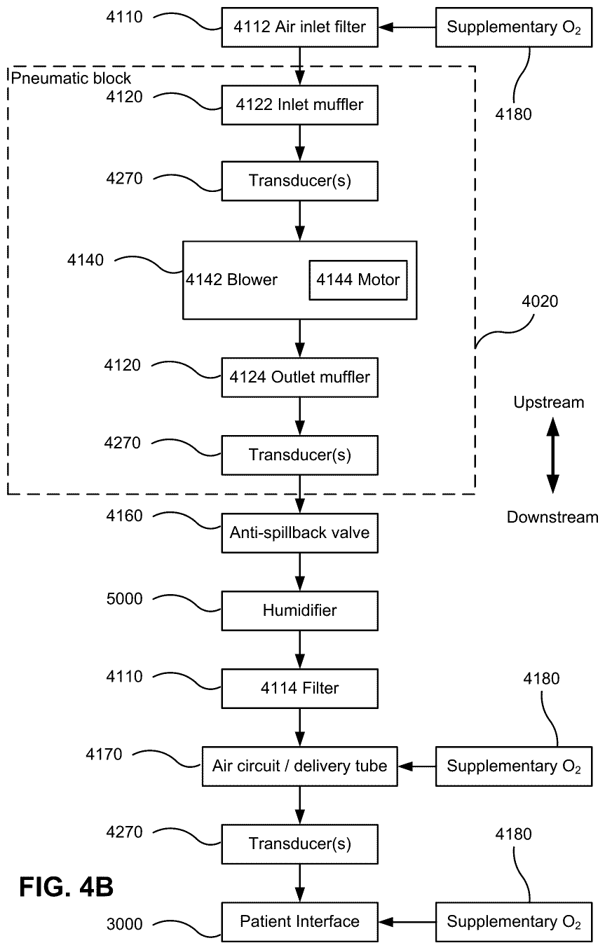

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 3.5 Humidifier

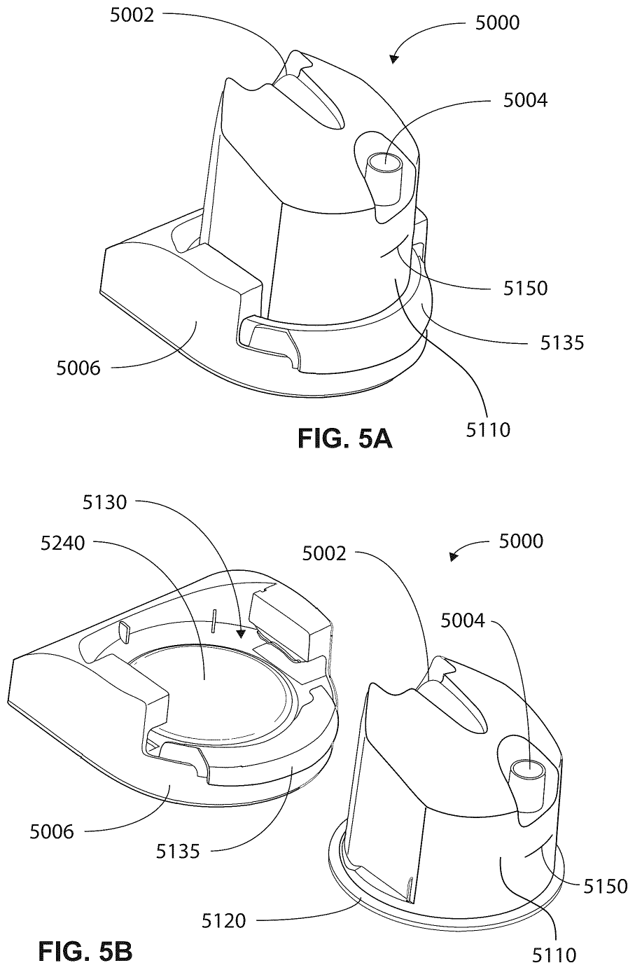

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 3.6 Heat Moisture Exchanger

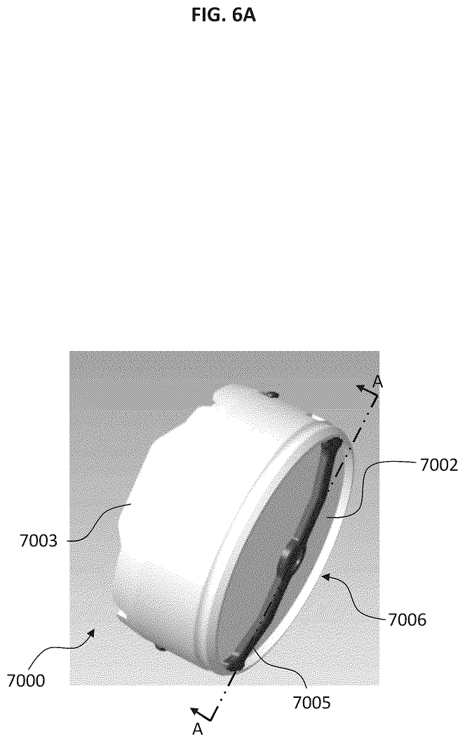

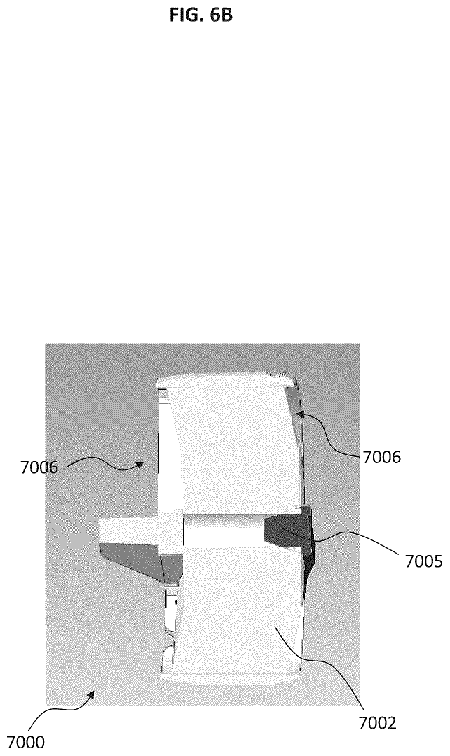

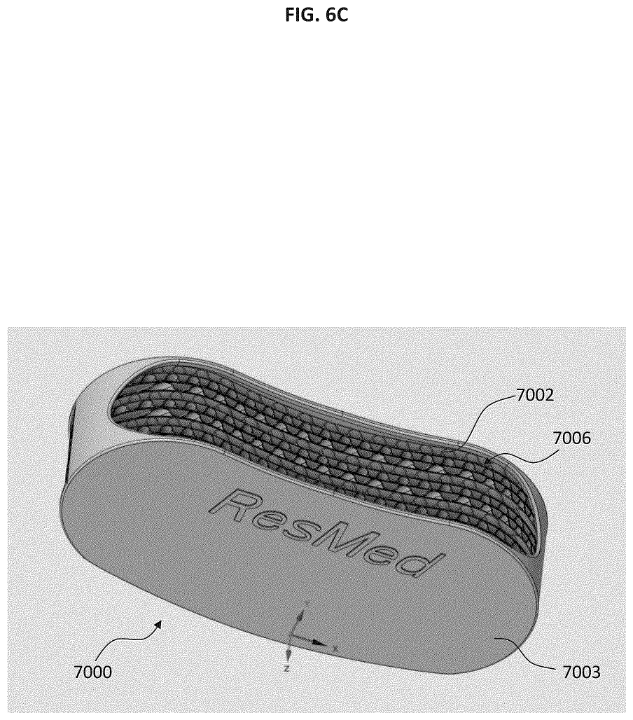

FIG. 6A shows an isometric view of a heat moisture exchanger in accordance with the present technology.

FIG. 6B shows a cross-sectional side view of the heat moisture exchanger of FIG. 6A taken through a mid-plane of the heat moisture exchanger, which is substantially parallel with the axis of the clip 7005 shown broadly as cross-sectional line A-A.

FIG. 6C shows an isometric view of a further heat moisture exchanger in accordance with the present technology.

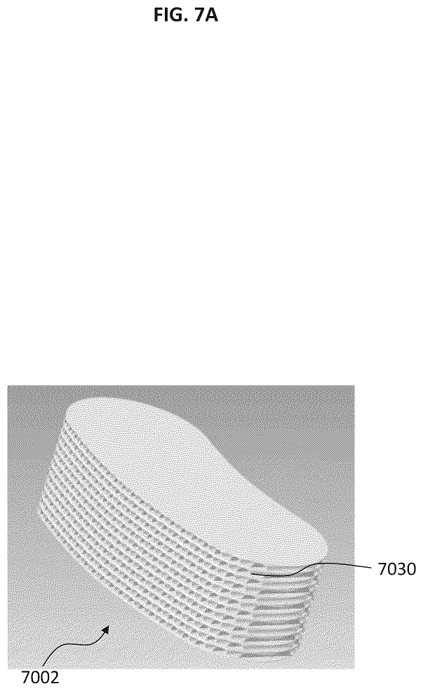

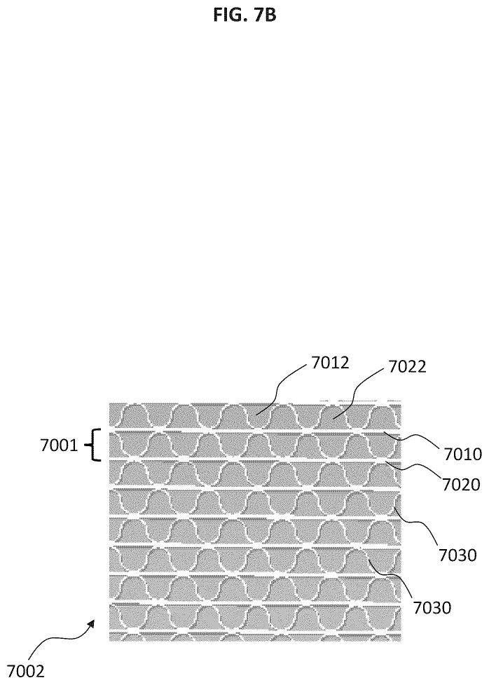

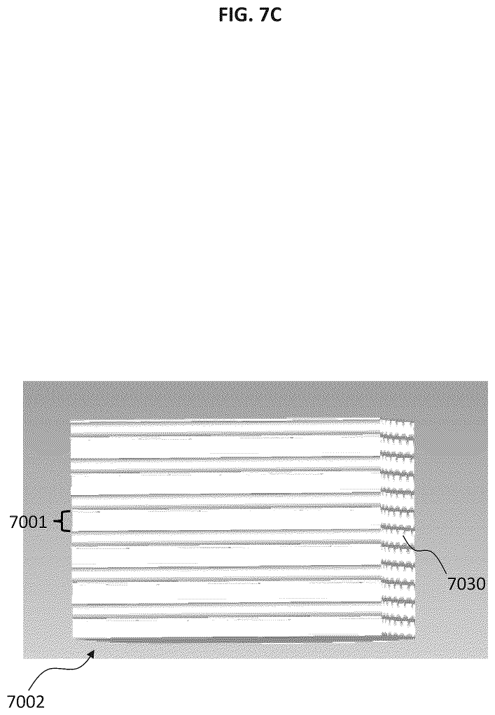

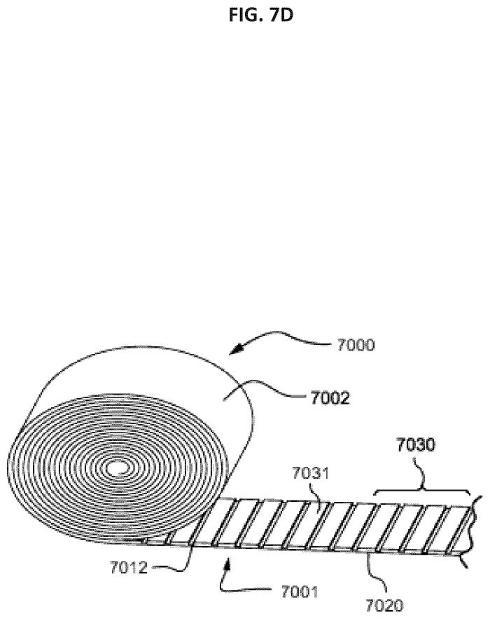

FIG. 7A shows an isometric view of an exemplary exchanging material 7002 construction for a heat moisture exchanger in accordance with the present technology.

FIG. 7B shows a front view of the exchanging material 7002 of FIG. 7A.

FIG. 7C shows a side isometric view of the exchanging material 7002 of FIG. 7A.

FIG. 7D shows an example isometric view of an alternative exchanging material 7002 in accordance with the present technology.

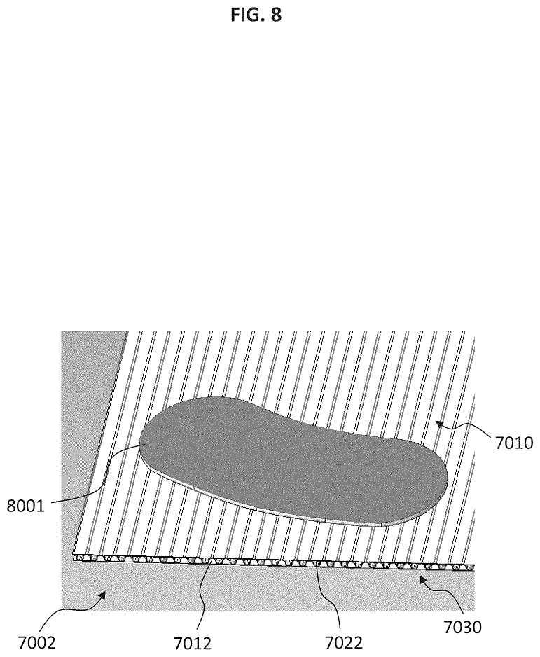

FIG. 8 shows an example of a process of creating a layer for a heat moisture exchanger from a sheet of corrugated material.

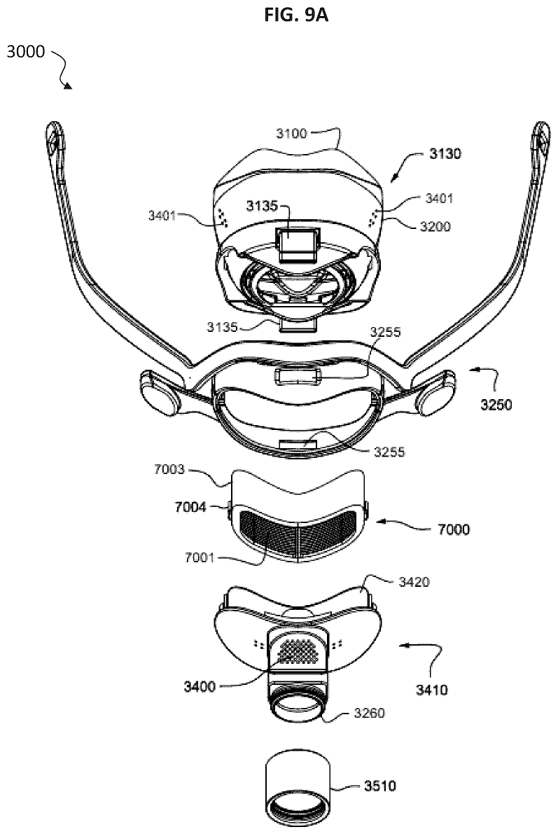

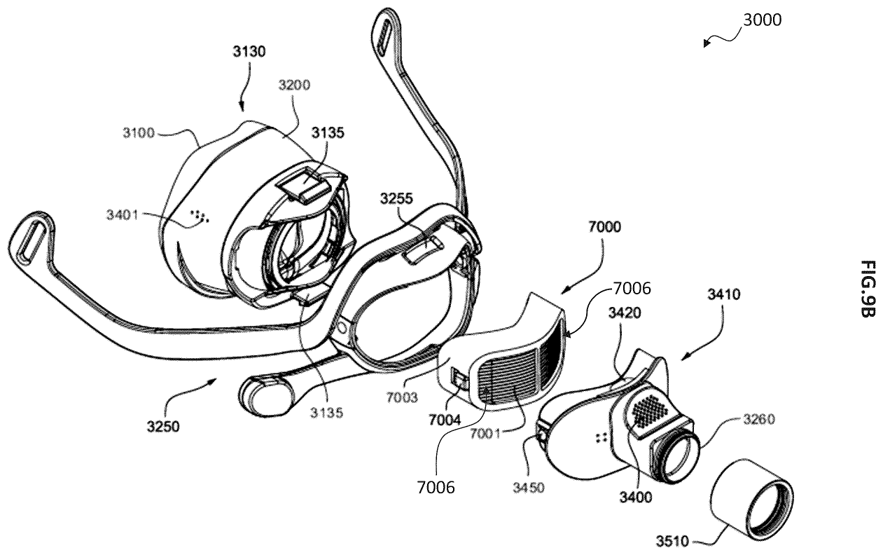

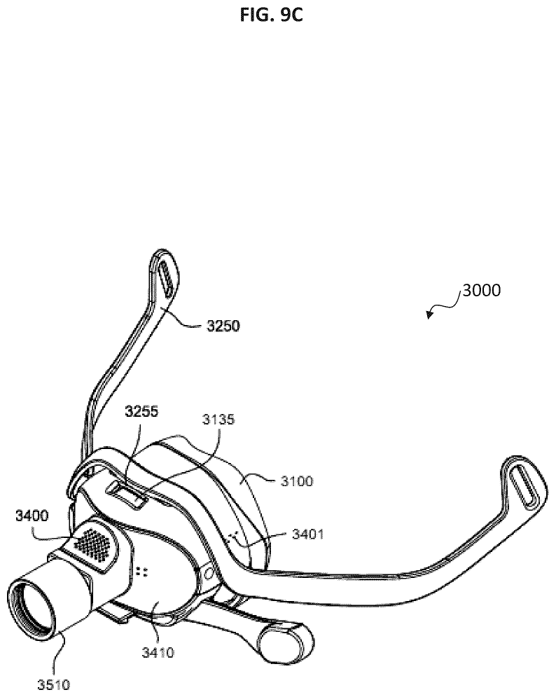

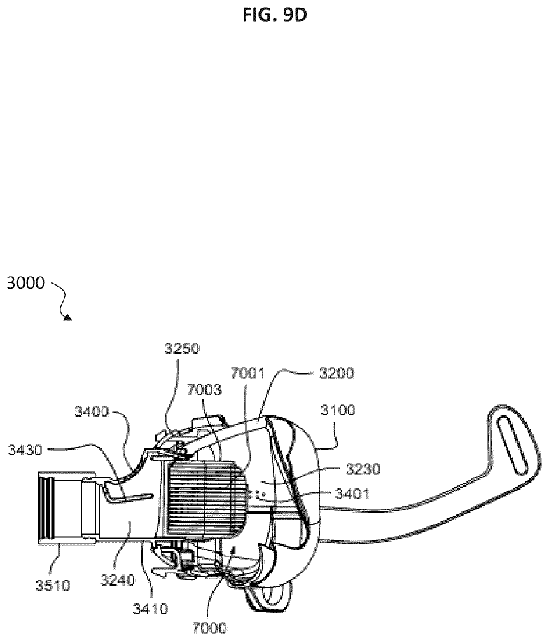

FIG. 9A shows a top exploded view of a patient interface comprising a heat moisture exchanger in accordance with the present technology

FIG. 9B shows an isometric view of the patient interface and heat moisture exchanger of FIG. 9A.

FIG. 9C show an assembled isometric view of the patient interface of FIG. 9A.

FIG. 9D shows a cross-sectional side view of the assembled patient interface of FIG. 9C, in which the cross section is taken through a plane which substantially cuts through the symmetric centre of the patient interface.

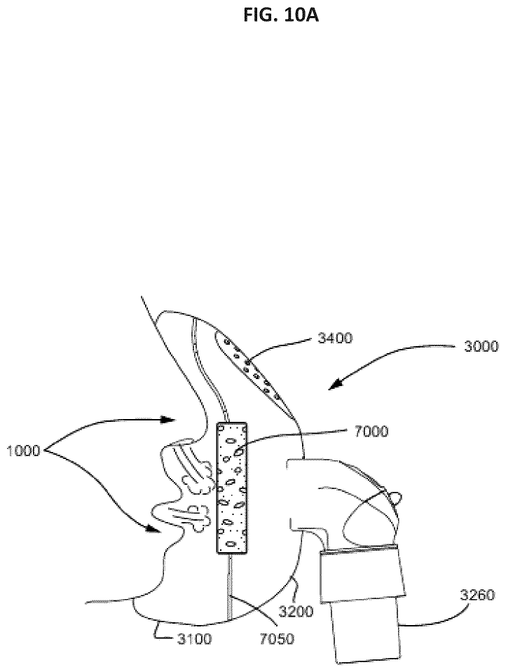

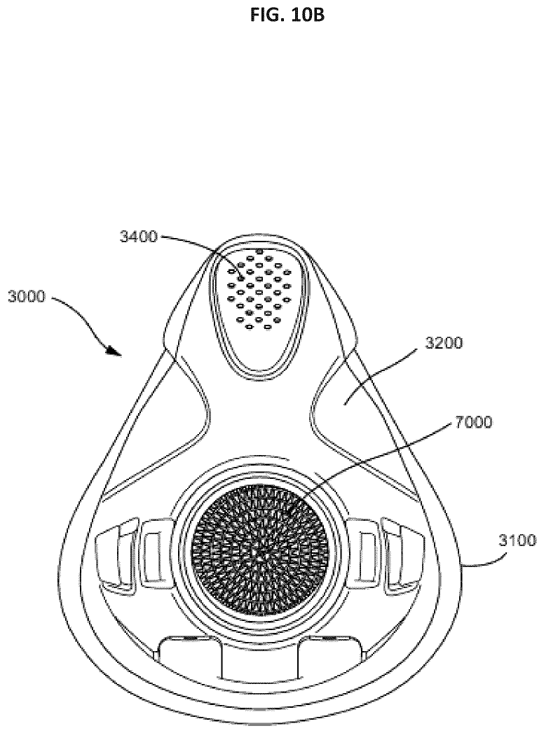

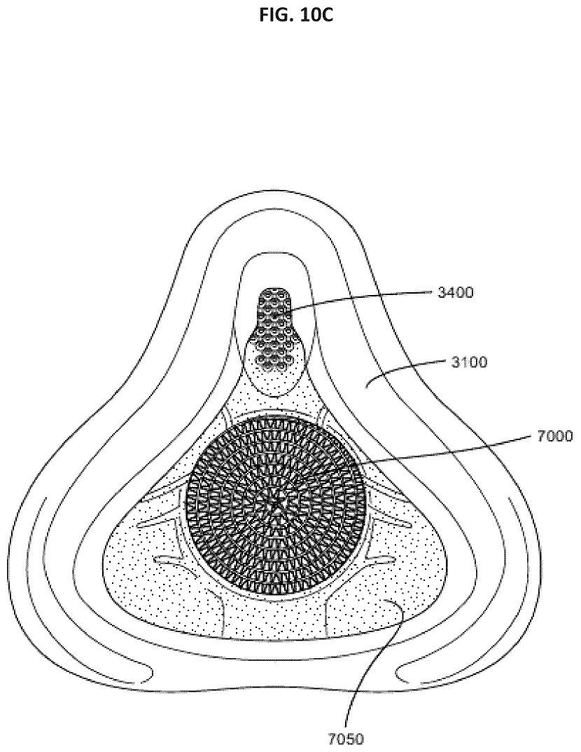

FIG. 10A is a side view of a patient interface 3000 with an HME 7000 positioned within the patient interface.

FIG. 10B is a front view of a patient interface 3000 comprising a HME 7000.

FIG. 10C is a rear view of a patient interface 3000 comprising a HME.

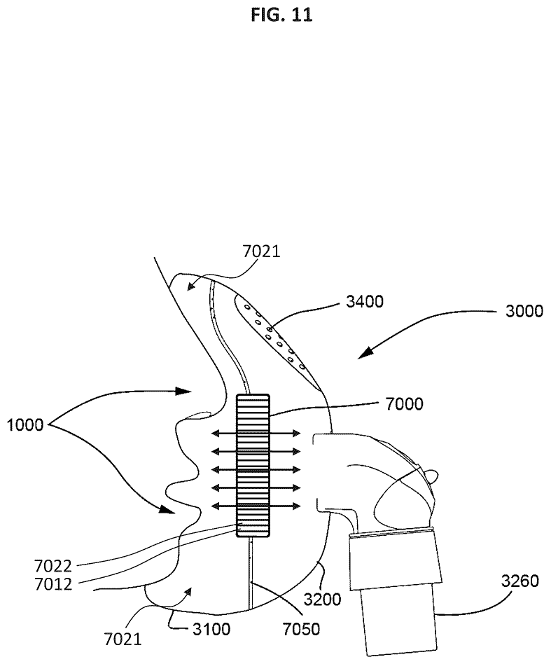

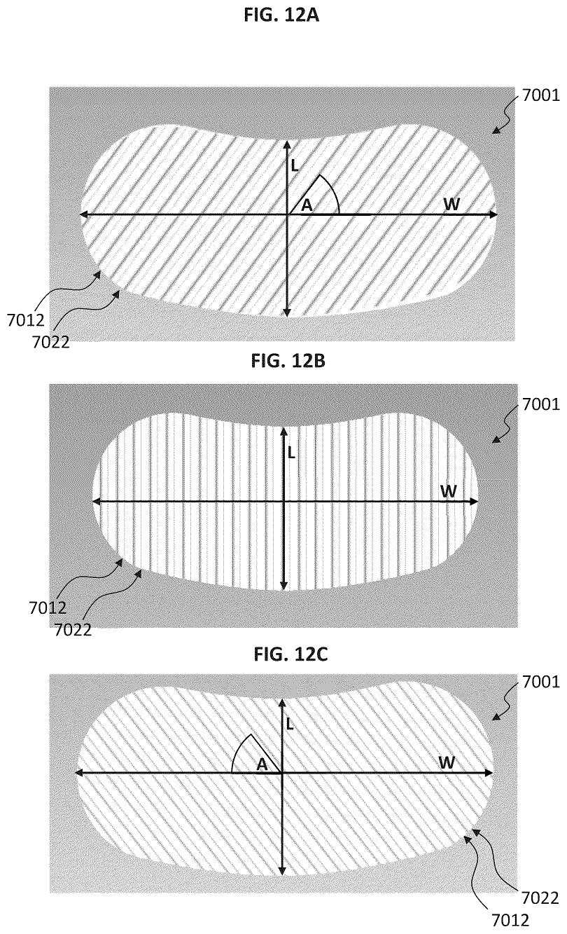

FIG. 11 is a side view of a patient interface 3000 with an HME 7000 positioned within the patient interface.

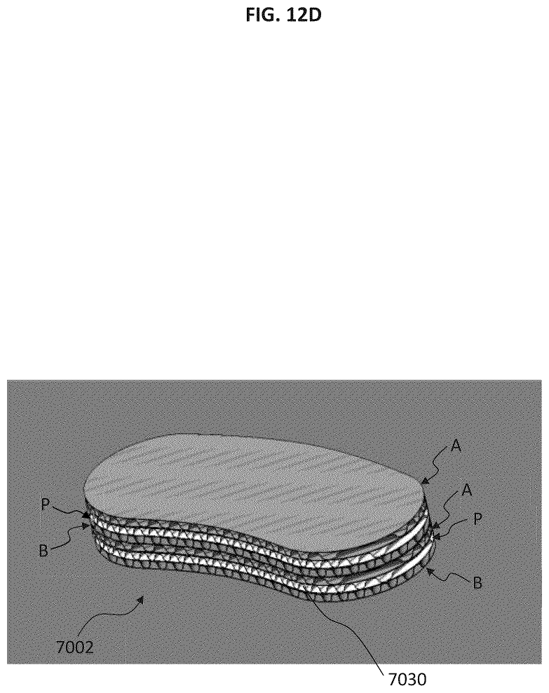

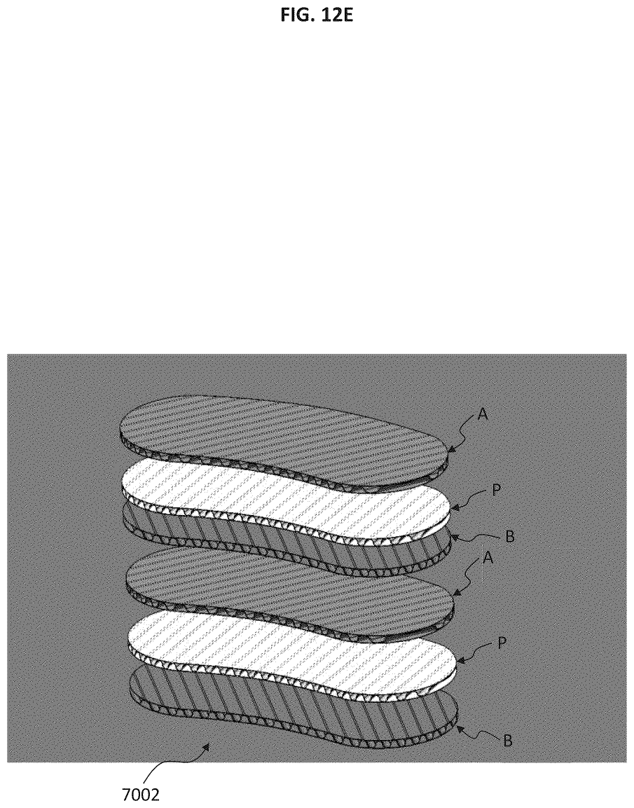

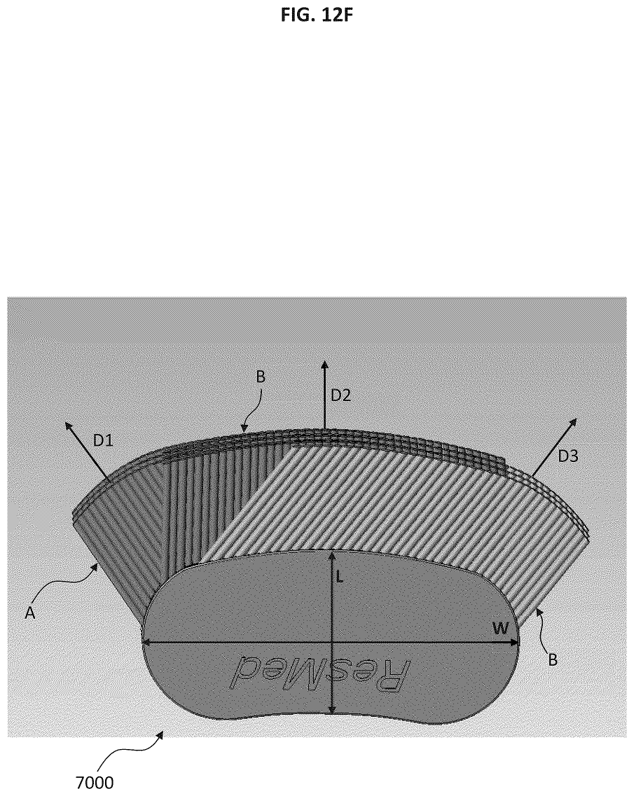

FIG. 12A is a top view of an exchanging material 7002 layer 7001 in accordance with the present technology.

FIG. 12B is a top view of an exchanging material 7002 layer 7001 in accordance with the present technology.

FIG. 12C is a top view of an exchanging material 7002 layer 7001 in accordance with the present technology.

FIG. 12D is an isometric view of an assembled HME 7000 exchanging material 7002 in accordance with the present technology.

FIG. 12E is an exploded view of the HME 7000 exchanging material 7002 of FIG. 12D.

FIG. 12F shows a HME 7000 where the exchanging material is shown to extend outwardly of the housing for illustrative purposes only.

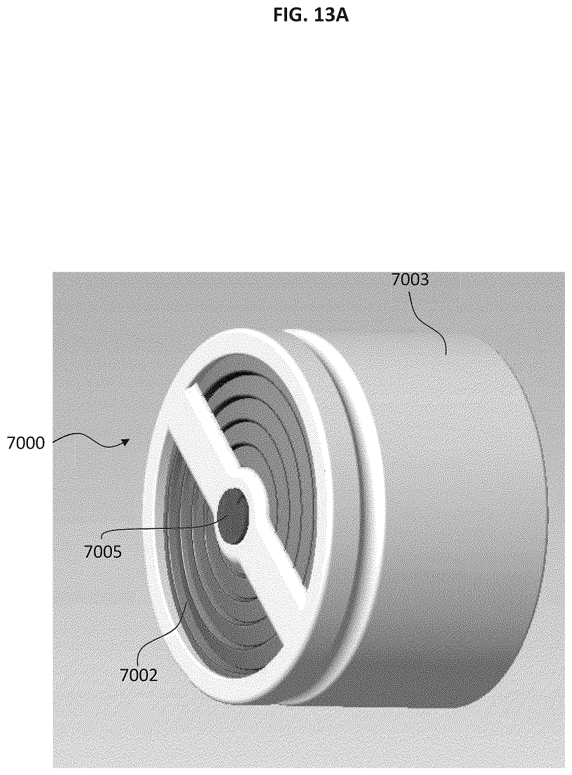

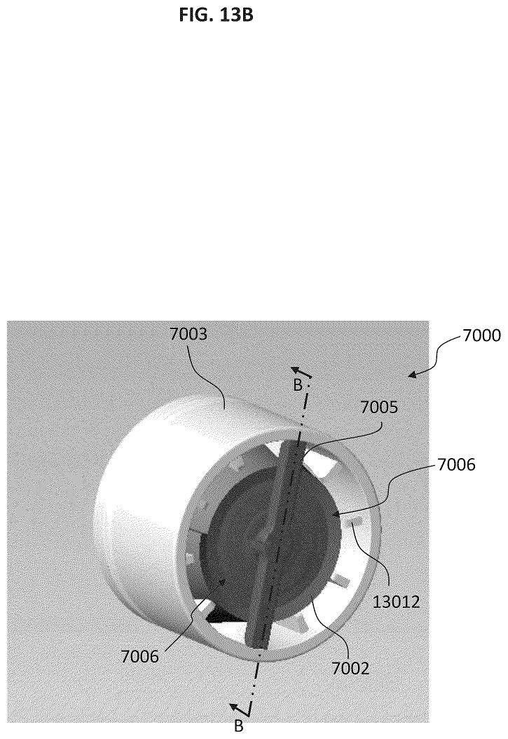

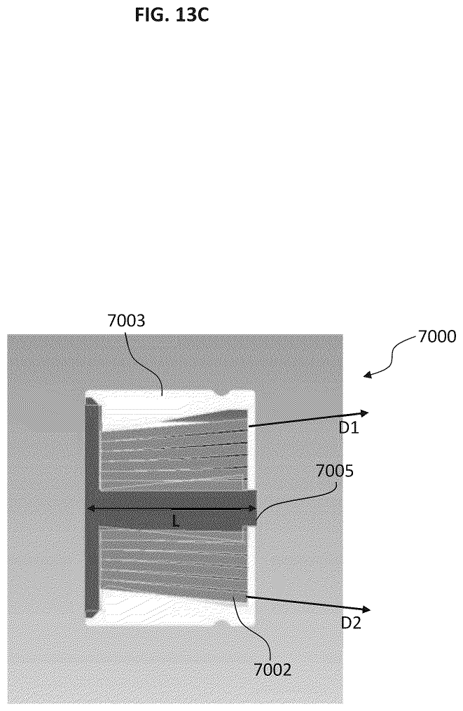

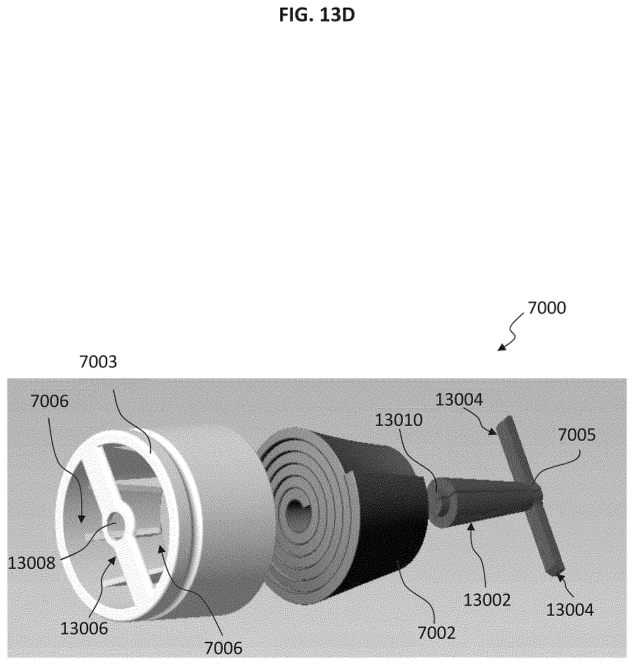

FIG. 13A shows a front isometric view of a cylindrical HME 7000 constructing according to a further aspect of the technology.

FIG. 13B shows a rear isometric view of the cylindrical HME 7000 of FIG. 13A.

FIG. 13C shows a cross-sectioned side view of the cylindrical HME of FIG. 13A, wherein the cross section is taken through the section lines marked as B-B in FIG. 13B.

FIG. 13D is an exploded isometric view of the cylindrical HME 7000 of FIG. 13A.

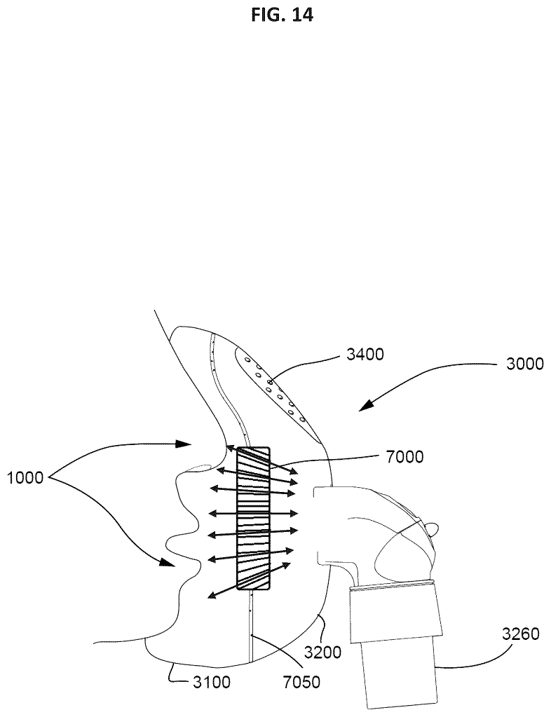

FIG. 14 is a schematic side view of a patient interface comprising an HME 7000 in accordance with the present technology.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 4.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

An unsealed patient interface 3800, in the form of a nasal cannula, includes nasal prongs 3810a, 3810b which can deliver air to respective nares of the patient 1000 via respective orifices in their tips. Such nasal prongs do not generally form a seal with the inner or outer skin surface of the nares. This type of interface results in one or more gaps that are present in use by design (intentional) but they are typically not fixed in size such that they may vary unpredictably by movement during use. This can present a complex pneumatic variable for a respiratory therapy system when pneumatic control and/or assessment is implemented, unlike other types of mask-based respiratory therapy systems. The air to the nasal prongs may be delivered by one or more air supply lumens 3820a, 3820b that are coupled with the nasal cannula-type unsealed patient interface 3800. The lumens 3820a, 3820b lead from the nasal cannula-type unsealed patient interface 3800 to a respiratory therapy device via an air circuit. The unsealed patient interface 3800 is particularly suitable for delivery of flow therapies, in which the RPT device generates the flow of air at controlled flow rates rather than controlled pressures. The “vent” or gap at the unsealed patient interface 3800, through which excess airflow escapes to ambient, is the passage between the end of the prongs 3810a and 3810b of the nasal cannula-type unsealed patient interface 3800 via the patient's nares to atmosphere.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure above the ambient.

The patient interface 3000 in accordance with one form of the present technology is constructed and arranged to be able to provide a supply of air at a positive pressure of at least 2 cmH2O with respect to ambient.

The patient interface 3000 in accordance with one form of the present technology is constructed and arranged to be able to provide a supply of air at a positive pressure of at least 4 cmH2O with respect to ambient.

The patient interface 3000 in accordance with one form of the present technology is constructed and arranged to be able to provide a supply of air at a positive pressure of at least 6 cmH2O with respect to ambient.

The patient interface 3000 in accordance with one form of the present technology is constructed and arranged to be able to provide a supply of air at a positive pressure of at least 10 cmH2O with respect to ambient.

The patient interface 3000 in accordance with one form of the present technology is constructed and arranged to be able to provide a supply of air at a positive pressure of at least 20 cmH2O with respect to ambient.

### 4.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

### 4.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

### 4.3.1.2 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

### 4.3.1.3 Nose-Only Masks

In one form, the patient interface 3000 comprises a seal-forming structure 3100 configured to seal around an entrance to the patient's nasal airways but not around the patient's mouth. The seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 1B has this type of seal-forming structure 3100. This patient interface 3000 may deliver a supply of air or breathable gas to both nares of patient 1000 through a single orifice.

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In one form the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

In some forms, a nose-only mask may comprise nasal pillows, described above.

### 4.3.1.4 Nose and Mouth Masks

In one form, the patient interface 3000 comprises a seal-forming structure 3100 configured to seal around an entrance to the patient's nasal airways and also around the patient's mouth. The seal-forming structure 3100 may be configured to seal to the patient's face proximate a chin region. This patient interface 3000 may deliver a supply of air or breathable gas to both nares and to the mouth of patient 1000. This type of patient interface may be identified as a nose and mouth mask.

One form of nose and mouth mask according to the present technology is what has traditionally been identified as a “full-face mask”, having a seal-forming structure 3100 configured to seal on the patient's face around the nose, below the mouth and over the bridge of the nose. A full-face mask may be generally triangular in shape. In one form the patient interface 3000 comprises a seal-forming structure 3100 that forms a seal in use to a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “full face cushion”.

In another form the patient interface 3000 comprises a seal-forming structure 3100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may have a nasal portion and an oral portion, the nasal portion sealing to the patient's face at similar locations to a nasal cradle mask.

In a further form of nose and mouth mask, the patient interface 3000 may comprise a seal-forming structure 3100 having a nasal portion comprising nasal pillows and an oral portion configured to form a seal to the patient's face around the patient's mouth.

In some forms, the seal-forming structure 3100 may have a nasal portion that is separate and distinct from an oral portion. In other forms, a seal-forming structure 3100 may form a contiguous seal around the patient's nose and mouth.

It is to be understood that the above examples of different forms of patient interface 3000 do not constitute an exhaustive list of possible configurations. In some forms a patient interface 3000 may comprise a combination of different features of the above described examples of nose-only and nose and mouth masks.

### 4.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

### 4.3.3 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position. Examples of a positioning and stabilising structure may be shown in FIG. 3A.

In one form the positioning and stabilising structure 3300 provides a retention force at least sufficient to overcome the effect of the positive pressure in the plenum chamber 3200 to lift off the face (i.e., Fplenum).

In one form the positioning and stabilising structure 3300 provides a retention force to overcome the effect of the gravitational force on the patient interface 3000.

### 4.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

### 4.3.5 Decoupling Structure(s)

In one form the patient interface 3000 includes at least one decoupling structure, for example, a swivel or a ball and socket.

### 4.3.6 Connection Port

Connection port 3600 allows for connection to the air circuit 4170.

4.3.7 Forehead Support In one form, the patient interface 3000 includes a forehead support 3700.

### 4.3.8 Anti-Asphyxia Valve

In one form, the patient interface 3000 includes an anti-asphyxia valve.

### 4.4 Rpt Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 4 cmH2O, or at least 10 cmH2O, or at least 20 cmH2O.

### 4.4.1.1 Pressure Generator

In one form of the present technology, a pressure generator 4140 for producing a flow, or a supply, of air at positive pressure is a controllable blower 4142. For example, the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH2O to about 20 cmH2O, or in other forms up to about 30 cmH2O when delivering respiratory pressure therapy. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

### 4.5 Air Circuit

An air circuit 4170 in accordance with an aspect of the present technology is a conduit or a tube constructed and arranged to allow, in use, a flow of to travel between two components such as RPT device 4000 and the patient interface 3000 or 3800.

### 4.6 Humidifier

### 4.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 4.7 Heat Moisture Exchangers

### 4.7.1 Overview

FIGS. 6A and 6B show one example of a HME 7000 according to the present technology, while FIG. 6C shows an alternative construction of a HME according to another form of the present technology.

In each example, the HME 7000 comprises an exchanging material 7002 which is generally provided as a corrugated or foam material. This exchanging material 7002 is configured to at least partially exchange heat and/or moisture present in exhaled gas from a patient's airways with the flow of breathable gas from a flow generator prior to inspiration. Various examples of HME constructions are provided in PCT Publication No. WO2015013761A1 which is herein incorporated in its entirety by reference.

The HME 7000 may generally comprise a HME frame 7003 which at least partially contains the exchanging material 7002. For example, in some aspects of the technology, the exchanging material may be contained entirely within the HME frame 7003, while in other aspects, the exchanging material may be partly contained within the HME frame 7003 and extend at least partially externally to the HME frame 7003.

In some examples of the technology the exchanging material 7002 may be retained within the HME frame 7003 by a retention mechanism, for example by using a clip 7005. For example, the clip may releasably attach to the HME frame 7003 to retain the exchanging material 7002 at least partially within the HME frame 7003. For example, the clip 7005 may releasably attach to the HME frame 7003 using any suitable fastener, including clips, screws, and adhesives.

In some examples of the technology, it can be advantageous for the exchanging material 7002 to be releasably connectable to the HME frame 7003 so that the exchanging material 7002 may be removed and replaced as required. For example, the clip 7005 may be releasably connected to the frame 7003 such that removal of the clip allows for removal and replacement or cleaning of the exchanging material 7002.

The HME 7000 comprises a plurality of openings 7006 through which the flow of breathable gas flows in use. For example, the plurality of openings 7006 can be configured to provide a pathway for a supply of breathable gas from the RPT device 4000 through the HME 7000 in a first direction, to the airways of the patient, and from the airways of the patient through the HME 7000 in a second direction to the vent 3400 during expiration from the patient. It should be appreciated that the first direction may be substantially opposite to the second direction, however this should not be seen as limiting, for example the expired air may take a return path which is different in one or more respects from the inhaled air.

For example, the openings 7006, may include a first opening 7006 on a first side of the HME 7000 and a second opening 7006 on a second side of the HME 7000. The positioning of the first opening relative to the second opening may generally define a longitudinal axis of the HME. For example, the longitudinal axis may extend perpendicularly between the two openings, and central to a width of the HME 7000.

FIGS. 7A to 7C show an example of an exchanging material 7002 with a corrugated structure. In this example the corrugated structure comprises a plurality of layers 7001, where each of the layers 7001 comprises corrugations 7030 between a substantially planar substrate top structure 7010 and a substantially planar substrate base structure 7020. In some aspects of the technology, these layers are stacked on top of one another to provide an HME 7000 with a multi-layered exchanging material.

Each layer 7001 comprises a plurality of channels 7012 through which the breathable gas flows during use of the HME 7000. In the example of FIGS. 7A to 7C, the channels 7012 are formed between the corrugations 7030 and the top structure 7010 and as well as between the corrugations and the bottom structure 7020. In this way, channels 7012 are formed on both sides of the corrugations 7030. This example however should not be seen as limiting, for example the channels 7012 may be formed on a single side of the corrugations 7030, or in examples of the technology comprising non-corrugate exchanging material 7002 such as foams, paper and textiles, the channels may be formed using any suitable techniques including material layering, moulding, shaping, puncturing or drilling. For example, the channels 7012 may be formed by puncturing a plurality of channels or holes in the exchanging material, or moulding or shaping the exchanging material around a removable core, wherein removing the core forms the channels.

FIG. 8 shows one aspect of the technology where the HME layers 7001 may be provided from a substantially planar sheet of corrugated material 7030. For example, by using a template 8001 and cutting (such as die-cutting, stamping, laser cutting, water-jet cutting, CNC routing etc.) each layer from the sheet of corrugated material 7030. In software controlled cutting examples, the template 8001 may be provided in software, while in die-cutting and stamping examples the template may be a physical template configured to define the shape of the cut. It should be appreciated that the template 8001 may be moved and rotated as necessary to form a plurality of layers having different channel angles as described herein.

Accordingly, one form of the technology provides a method of manufacturing layers 7001 of a HME 7000 by cutting a plurality of layers from one or more substantially flat planar sheets of an exchanging material. In some forms of the technology the method comprises cutting a plurality of layers from one or more substantially flat planar sheets of exchanging material, wherein one or more of the plurality of layers has a different channel angle to one or more of the other of the plurality of layers.

It should be appreciated that in aspects of the technology where a plurality of layers 7001 are used, the top structure 7010 of one layer 7001 may be the same as or otherwise attached to the base structure 7020 of the adjacent layer 7001 and vice versa.

The HME 7000 allows for a flow of breathable gas and expiratory gas to flow through the plurality of channels 7012 along a surface of the exchanging material 7002 to exchange heat and moisture. Moisture is absorbed from the expiratory gas exhaled from a patient and retained in the exchanging material 7002 such as the corrugated structure shown in FIGS. 7A to 7C. The material of the corrugations 7030, the top structure 7010, and/or the base structure 7020 may comprise foam, paper or a paper-based material that is able to absorb water and/or heat. The material of the corrugations 7030, the top structure 7010, and/or the base structure 7020 may be porous, water-permeable, and/or air-permeable. The retained moisture may subsequently be redelivered to the patient by humidifying a flow of breathable gas delivered to the patient's airways. In other words, the flow of breathable gas delivered to the patient's airways may absorb moisture from the HME 7000.

The plurality of corrugations 7030 provide an increased surface area of the exchanging material 7002 when compared to some non-corrugated structures, such as when using layers of exchanging material separated by rectangular or square channels 7012. This increased surface area may allow for an increase in active surface area for the exchange of heat and moisture occurring between the exchanging material 7002 and the surrounding volume provided by the plurality of channels 7012. The top structure 7010 and the base structure 7020 may also be formed from the same heat and moisture exchanging material as the corrugated structure 7030. Alternatively, the top structure 7010 and/or the base structure 7020 may be formed of a rigid or semi-rigid material that does not absorb moisture to support the corrugated structure 7002.

One of the factors affecting humidification performance of the HME 7000 is the effective surface area of the HME 7000 provided in a fixed volume of space. The effective surface area is the surface area of the HME 7000 that is exposed to the flow of breathable gas flowing along the surface of the HME where heat and moisture exchange occurs. The surface area per unit volume of the HME 7000 can be adjusted by providing corrugations 7030 within the heat and moisture exchange portion of the HME 7000. Furthermore, the surface area per unit volume may also be adjusted by modifying at least one of the fin thickness, pitch or height of the corrugations or flutes, which have an impact on the surface area per unit volume of the HME 7000. Further examples of how the corrugation or fin thickness, pitch or height of the corrugations may be adjusted is described in detail in PCT Publication No. WO2015013761A1 which is herein incorporated in its entirety by reference.

The HME 7000 may comprise a plurality of layers 7001 stacked along an axis of the HME 7000, as shown in FIG. 7C. The layers 7001 may be stacked such that the base structure 7020 is stacked on top of the corrugated structure 7002 of an underlying adjacent layer 7001. Having a plurality of layers 7001 comprising corrugated structures 7002 that are stacked along an axis of the HME 7000 can further increase the surface area per unit volume of the HME when compared to HME constructions which utilise a single layer of exchanging material 7002. This increased surface area within a predefined volume allows for increased efficiency in heat and moisture exchange of the HME 7000.

As shown in FIG. 7D, displaying an alternative example form of the technology, the HME 7000 may be rolled from a single strip layer 7001 comprising a corrugated structure 7002 extending from the surface of the base structure 7020 to form a plurality of corrugations 7030 comprising a plurality of channels 7012. The single strip layer 7001 may be rolled such that the upper folded portion 7031 of the corrugations 7030 engages the inferior surface of the base structure 7020. This configuration ensures that the plurality of channels 7012 is maintained between each roll of the single strip layer 7001.

The HMEs 7000 described herein may be positioned within a plenum chamber 3200 of the patient interface 3000, or otherwise in the airflow pathway between the RPT device 4000 and the patient's airways.

It should be appreciated that the foregoing example should not be seen as limiting on the technology, and any suitable exchanging material 7002 construction may be used, including any substance capable of acting as a condensation and absorption surface such as corrugated materials, foams, paper, textile and composite materials. In some examples of the technology the exchanging material may carry hygroscopic salts to improve the water-retaining capacity. Suitable salts include calcium chloride.

### 4.7.2 Patient Interfaces

In an example of a non-invasive patient interface 3000 in accordance with one aspect of the present technology, the patient interface 3000 may comprise the following functional aspects: a seal-forming structure 3100, a plenum chamber 3200, a HME 6000 positioned in the functional dead space within the plenum chamber 3200, a supporting membrane 7050 structure to hold the HME 7000 in position, a positioning and stabilising structure 3300 and a connection port or inlet 3260 for connection to air circuit 4170. In some forms a functional aspect may be provided by one or more physical components. In some forms, one physical component may provide one or more functional aspects. In use the seal-forming structure 3100 is arranged to surround an entrance to the airways of the patient so as to facilitate the supply of air at positive pressure to the airways.

A positioning and stabilising structure 3300 may be provided to releasably secure the patient interface 3000 to the patient 1000 as shown in FIG. 3A. The positioning and stabilising structure 3300 may include a plurality of straps that are adjustable in length to allow the patient interface to be comfortably and securely fitted to the patient 1000 such that a pneumatic seal is formed around the patient's airways by the seal-forming structure 3100. A strap connector 3301 may also be provided to releasably secure the straps of the positioning and stabilising structure 3300 to the patient interface 3000. The straps of the positioning and stabilising structure 3300 may include hook and loop material for length adjustment and to allow the straps of the positioning and stabilising structure 3300 to be attached to and detached from the strap connector 3301. It should be understood that the strap connector 3301 may be releasably attached to the patient interface 3000 or it may be integrally formed therewith.

FIGS. 9A to 9D illustrate an example of the technology wherein a HME 7000 is provided to a patient interface 3000. The patient interface 3000 in this example has cushion assembly 3130, which is optionally provided with a plurality of cushion assembly engagement members 3135 in the form of a clip comprising a resilient flange. These cushion assembly engagement members 3135 provide a means to removably engage the cushion assembly to a mask frame 3250. The mask frame 3250 comprises a mask frame engagement member 3255 in the form of a recess or hole that allows for the resilient flange of the cushion assembly engagement member 3135 to pass and removably engage thereto. Alternatively, the cushion assembly 3130 may engage to the mask frame by other methods such as hook, adhesive, interference or frictional engagement.

The cushion assembly 3130 comprises a seal forming structure 3100. The seal forming structure 3100 may form a seal with the entrance of a patient's airways. In addition, the seal-forming structure 3100 of the patient interface 3000 may comprise a pair of nasal puffs, or nasal pillows, each nasal puff or nasal pillow being constructed and arranged to form a seal with a respective naris of the nose of a patient. Alternatively, the seal forming structure may form a seal with the nares and the mouth.

The exemplary patient interface 3000 further comprises a removable HME 7000 that removably engages with the patient interface 3000 and the HME 7000 may be located within a HME housing portion 3420 of a vent adaptor 3410. The HME 7000 may comprise at least one HME engagement member 7004 positioned on the HME frame 7003. The at least one HME engagement member 7004 may comprise at least one clip and each of which may removably engage a corresponding vent adaptor engagement member 3415. The vent adaptor 3410 may comprise a vent 3400 and a mask inlet 3260 positioned on its anterior side. The vent adaptor 3410 may be adapted to removably engage to the remainder of the patient interface 3000 and locate the removably engageable HME 7000 within its HME housing portion 3420. The vent adaptor 3410 may locate the HME 7000 in a flow path of breathable gas within a plenum chamber 3200 of the patient interface 3000 and may orient the plurality of channels 7012 of the HME to be substantially in line with or parallel to a flow path of the flow of breathable gas, thereby allowing flow through the HME 7000 via the channels 7012. The positioning of the HME 7000 in close proximity to the entrance of the patient's airways may maximise the capture and retention of humidity that is provided to the material of the HME 7000 during exhalation. Moreover, the orientation of the channels 7012, may also allow the flow of humidified gas exhaled from the patient to flow through the channels 7012 of the HME 7000 in the opposing direction.

The vent adaptor 3410 may locate the HME 7000 within or adjacent to the plenum chamber 3200 and may divide said plenum chamber 3200 into two chambers, for example an anterior plenum chamber which includes the HME 7000 and a posterior plenum chamber configured to receive the patient's nose and or mouth in use. The HME 7000 may be configured to locate the vent 3400 and inlet 3260 on an anterior side of the HME 7000 as part of the anterior plenum chamber 3240 with the entrance of the patient's airways on a posterior side of the HME 7000, adjacent to the posterior plenum chamber 3230. This configuration may allow the flow of exhaled gas from the patient to flow into the posterior plenum chamber 3240 prior to venting, which allows any humidity to be retained in the HME 7000 prior to losses out of the vent 3400. Furthermore, the configuration also may allow the flow of breathable gas to flow through the HME 7000 prior to redelivery of the captured humidity to the patient. Thus, the housing portion 3410 may provide a configuration for redelivering humidified air to a patient via a HME 7000 positioned in the flow path of the patient interface 3000.

The vent adaptor 3410 may also include connectors configured to connect to the HME frame 7003. For example, using a snap-fit connection.

In some forms the patient interface may additionally comprise an auxiliary vent 3401 on the posterior side of the HME in the posterior plenum chamber 3240 to offset CO₂ build up within this volume. For example, in the case of a full-face mask, the additional volume in the posterior plenum chamber 3240 (i.e., dead space volume) in comparison to smaller masks, may lead to unwanted and/or excessive CO₂ build up occurring within this space. To mitigate this effect, it is possible to position an auxiliary vent 3401 proximal to the patient's airways, on the posterior or patient side of the HME 7000. Positioning an auxiliary vent 3401 on the posterior side of the HME 7000 results in some venting of the humidified flow of breathable gases prior to delivery to the patient. To compensate for this venting of humidified air, the overall humidification performance may be maintained by increasing the ability of the HME 7000 to humidify the flow of breathable gas within a predetermined volume of the plenum chamber 3400.

The vent adaptor 3410 may also include a baffle 3430 to separate the incoming flow of breathable gas from the flow of CO₂ washout. The baffle 3430 may separate these flows of gas from one another such that these flows of gas do not interfere with one another. U.S. Pat. No. 7,934,501, which is incorporated herein by reference in its entirety, describes further examples and features of baffles that may be applicable to the exemplary patient interface 3000.

In other examples of the technology described herein, one of more of the channels within the exchanging material may be configured to direct the expired air through the HME toward a vent.

The HME 7000 may be stacked in layers 7001 and further comprise a rigid supporting HME frame 7003. The HME layers 7001 may be retained within the HME frame 7003 by any techniques known to those skilled in the art, including fasteners, adhesives and by being secured in place due to being positioned within the HME frame 7003.

According to another aspect of the technology, as shown in FIGS. 10A to 10C, a HME 7000 may be provided within the functional dead space of a patient interface 3000. In other words, the HME may be provided in an area of the patient interface which is otherwise not occupied by the patient's facial features in use. In these examples, the HME 7000 has a substantially cylindrical construction, wherein the flow of breathable gas through the HME 7000 is substantially in direction of the longitudinal axis of the HME 7000. However, this should not be seen as limiting on the technology.

The HME 7000 may be positioned in the plenum chamber 3200 such that it remains between the patient's 1000 airways and the mask vent 3400/inlet 3260 of the patient interface 3000. The HME 7000 may be supported and held in position by a supporting membrane 7050 that may be connected to the inside walls of the plenum chamber 3200. The HME 7000 in these examples is circular in form and has a thickness of approximately 5-10 mm. Alternatively, the HME material may be moulded into a profiled shape which directly assembles to the interior profile of the plenum chamber 3200, wherein the HME make take on a shape complementary to the interior of the plenum chamber 3200. In this case the shape may be a three-dimensional surface with a thickness of approximately 1-10 mm.

Furthermore, while the example shown in FIGS. 10A to 10C shows the use of a cylindrical or circular HME in the internal space of the patient interface, this should not be seen as limiting and the HME 7000 may be positioned externally to the patient interface 3000, such as between the patient interface and an elbow, or between the elbow and the air delivery conduit.

### 4.7.3 Diffusing HME Constructions

One limitation of some existing HME constructions as illustrated in FIG. 11 is that, where corrugated exchanging materials 7002 are used, these tend to form relatively straight parallel pathways which the breathable gas passes directly through in use. This can cause discomfort to the patient as the incoming air is directed straight towards the patient's face and airways. This can result in a strong flow of breathable gas on the face of the user, particularly in examples of the technology where the HME 7000 is positioned within a patient interface 3000, as the patient's nose and/or mouth are often positioned close to the HME 7000. In addition, it can be important for the HME 7000 to be carefully positioned relative to the patient's airways within the patient interface 3000 in order to ensure that these types of HME 7000 function effectively, as the channels 7012 can result in restriction of the exhaled airflow if the channels are not in line with the patient's airways.

These types of HME 7000 also result in a relatively small projected area of air on the patient face, or being expelled from the HME 7000, as the parallel HME channels 7012 provide no diffusing of the incoming or outgoing airflow. In examples of the technology where the HME is external to the patient interface this can result in louder, and more direct venting of the expired air, which can cause disruption to the patient/user as well as any people nearby, such as those sleeping in the same bed.

Another potential issue is that there can be limited airflow within some dead space regions 7021 of the patient interface 3000 such as below, above, and/or to the sides of the patient's nose and/or mouth. The reduced air circulation within this area can result in an increased concentration of CO₂, which can be hazardous to the patient, or at least cause discomfort.

Accordingly, one aspect of certain forms of the technology provides a HME 7000 in which the airflow through the HME 7000 and/or the patient interface 3000 comprising the HME is relatively diffused when compared with existing HMEs 7000 and patient interfaces 3000. In some forms, the HME 7000 constructions described herein may be used for improving breathable gas circulation within a patient interface and/or CO₂ washout within patient interfaces irrespective of whether the structure used within the patient interface is configured for heat and/or moisture exchange.

In some forms, there is provided a heat moisture exchanger comprising a plurality of airflow channels through which a flow of breathable gas passes in use, and where at least one of the airflow channels is provided in a different orientation from at least one other airflow channel. In certain forms, the plurality of airflow channels may comprise sets of airflow channels where each set has a plurality of channels provided in the same orientation and where that orientation is different from the orientation of another set. In certain examples of this, as will next be described, the sets of airflow channels may be provided in layers. In other forms, the sets may be provided in other arrangements.

### 4.7.3.1 Multilayer Diffusion

FIGS. 12A to 12F show an example of the technology in which an HME 7000 is provided with a plurality of exchanging material 7002 layers 7001, each configured with a concertina construction. In this example, one or more of the exchanging material layers 7001 within the HME 7000 is provided with channels 7012, having different orientations to the channels of one or more of the other layers 7001 within the HME 7000.

For example, the HME 7000 may comprise one or more layers 7001, each with a plurality of channels 7012. Each layer 7001 has a length or longitudinal axis ‘L’ which, in use, extends in a particular direction relative to the face of the patient, for example perpendicular to the frontal (or coronal) plane (as shown in FIG. 2C) of the patient. The longitudinal axis may also be defined as an axis extending perpendicularly between a first opening 7006 on a first side of the HME and a second opening on a second side of the HME, the first and second openings defining ends of an airflow pathway through the HME.

The layers may each further have a lateral axis, or width ‘W’ which may be perpendicular to the longitudinal axis L. In use, the lateral axis may extend in a direction substantially perpendicular to the mid-sagittal plane of the patient, or otherwise in a direction substantially perpendicular to the axis between the openings 7006 of the HME 7000. The mid-sagittal plane should be familiar to those skilled in the art, and is illustrated in FIG. 2B.

One or more of the layers 7001 of the HME 7000 may comprise channels 7012, that are provided substantially parallel to the longitudinal axis ‘L’ of the layer, substantially parallel to the axis between the openings 7006, or substantially parallel to the direction of airflow between the patient interface 3000 and an end of the air circuit that connects to the HME, as shown in FIG. 12B. In other words, one or more of the layers may be provided with an angle of between 80 and 100 degrees of the longitudinal axis, or more preferably between 85 and 95 degrees, such as approximately 90 degrees. For simplicity this layer shall herein be referred to as a “parallel layer”, i.e. a layer in which the channels 7012, are substantially parallel to the longitudinal axis of the HME or the axis between the openings 7006 of the HME 7000. Use of at least one parallel layer may advantageously provide a direct, low flow-impedance path between the RPT device 4000 and the patient interface 3000 and/or airways of the patient.

In some examples of the technology, one or more of the layers 7001 of the HME 7000 may comprise channels 7012, that are provided at an angle ‘A’ relative the lateral axis ‘W’. This angle may alternatively be measured with respect to the longitudinal axis ‘L’ defined herein. For example, when the angle is defined with respect to the lateral axis ‘W’ or the longitudinal axis 1′ the angle may be between 30 and 60 degrees, or between 120 and 150 degrees. For simplicity this layer 7001 shall herein be referred to as an “angled layer”, i.e. a layer which is provided at a non-parallel angle relative to the direction of airflow between the openings 7006 of the HME 7000. Use of at least one angled layer may advantageously provide a more diffused airflow, and/or assist with washout of CO2 from the patient interface 3000.

In some examples of the technology, the HME 7000 comprises a first layer, and a second layer, wherein the channels in the first layer are provided at a first angle with respect to the lateral axis “W”, and the channels in the second layer are provided at a second angle with respect to the lateral axis ‘W’, where the first angle is different to the second angle. For example, the first layer may be a parallel layer, and the second layer may be an angled layer, or alternatively the first layer may be an angled layer, and the second layer may be an angled layer with a different channel angle to the first layer. The first angle and the second angle may be equal but opposite, for example +30 degrees and −30 degrees.

In some examples of the technology the HME 7000 comprises at least one parallel layer, a first angled layer in which the channels 7012, are provided at a first angle relative to the lateral axis ‘W’, and a second angled layer in which the which the channels 7012, are provided at a second angle relative to lateral axis ‘W’. For example, a plurality of parallel, first angled layers and second angled layers may be used.

In some examples of the technology, the first angle may be 60 degrees or less, and the second angle may be 120 degrees or more, when measured with respect to the lateral axis ‘W’.

FIG. 12D shows a stack of layers 7001 of a diffused HME 7000 in accordance with one form of the present technology. In this example the HME comprises two parallel layers “P”, two angled layers “A” configured with channels oriented in a first angle, and two angled layers “B” configured with channels oriented in a second angle. The first angle is different to the second angle, and the first angle may be approximately mirrored about the longitudinal axis of the diffusing material, mirrored about the axis between the openings 7006 of the HME, or mirrored about the direction of airflow between the patient interface and the RPT device 4000. In other words, the first angle and the second angle may advantageously sum to approximately 180 degrees, when measured with respect to the lateral axis ‘W’.

One potential advantage of using a “mirrored layer” is that, in applications of the technology where the exchanging material layers 7001 are substantially symmetrical about the direction of airflow between the patient interface and the RPT device 4000, the “mirrored layer” may be provided by simply flipping the opposing angled layers over, or otherwise rotating the layer 180 degrees through the longitudinal axis ‘L’. This can advantageously allow for simple cost-effective manufacturing of the HME 7000, for example as less inventory of layers may be required, when compared to using angled layers which are not mirrored.

This configuration, however, should not be seen as limiting on the technology. For example, each layer 7001 within the HME 7000 may be provided with different channel angles, or more layers may be provided with one channel angle than other layers. For example, a single layer may be provided with a first channel angle, and a plurality of parallel layers may be used.

It should be appreciated that the exact composition of channel angles within the HME 7000 may depend on the geometry of the patient interface 3000 that the HME is intended to be used with. For example, the channel angles ‘A’ may be configured to provide improved washout of CO2 from certain geometries of patient interfaces, such as more angled layers in examples where the patient interface comprises a greater volume of dead-space 7021 or a greater number of dead space 7021 areas within the interface. In some patient interfaces 3000, for example, it may be advantageous to use no “parallel” channels.

In the illustrated examples, the layers 7001 are oriented substantially perpendicular to the sagittal plane of the patient's face in use. The layers 7001 may also be oriented such that the top structure 7010 and bottom structure 7020 of one or more of the layers, for example of all the layers, are oriented approximately parallel to the Frankfort horizontal (see FIG. 2C) of the patient in use, although in some forms the patient interface may be configured with top and bottom structures oriented with a non-zero angle relative to the Frankfort horizontal, for example an acute angle. In this example the channel angles are configured to direct the flow of breathable gas from the RPT device 4000 to the left and/or right-hand sides of the patient's face in use. This configuration however should not be seen as limiting, for example the layers 7001 may be oriented substantially parallel to the sagittal plane of the patient's face, such that the lateral axis of the HME is substantially parallel to the sagittal plane. In this example of the technology the channel angles can be configured to direct the flow of breathable gas upwardly or downwardly on the patient's face in use.

In some examples of the technology, the HME 7000 may comprise one or more layers which are oriented in a substantially perpendicular to the sagittal plane of the patient's face in use, such that the lateral axis of the HME is substantially perpendicular to the sagittal plane of the user's face, and one or more layers which are oriented substantially parallel to the sagittal plane of the patient's face.

In other examples of the technology, the layers may not be oriented substantially parallel to, or perpendicular to the sagittal plane of the patient's face. For example, the layers may be provided with an arcuate or curved top structure 7010 or bottom structure 7020. In such examples, the intermediate structures between the layers may be similarly shaped. In other examples the top structure may be provided at a non-zero angle relative to the sagittal plane of the user's face, for example at an acute or obtuse angle with respect to the sagittal plane.

FIG. 12F shows an example of the technology wherein the channels of each of the respective layers are shown extending outwardly from the HME 7000 for purposes of illustrating the relative directions of the channels only. In this example, the direction of airflow provided by each of the respective layers can be identified. For example, a first angled layer ‘A’ is configured to direct the flow of air through the HME in a first direction ‘D1’ which is at a first angle with respect to the longitudinal axis ‘L’ of the HME 7000, a substantially parallel layer ‘P’ which is configured to direct the flow of air through the HME in a second direction D2, which is substantially parallel to the longitudinal axis ‘L’ of the HME 7000, and a second angled layer ‘B’ is configured to direct the flow of air through the HME in a third direction ‘D3’ which is at a second angle with respect to the longitudinal axis ‘L’ of the HME 7000.

As described herein the first angle may be substantially mirrored about the longitudinal axis ‘L’ of the HME. However, this should not be seen as limiting on the technology.

### 4.7.3.2 Spiral Diffusion

FIGS. 13A to 13D show an alternative HME 7000 construction in accordance with another form of the technology. The HME 7000 in FIGS. 13A to 13D comprises a substantially cylindrical HME frame 7003 and a rolled or otherwise substantially cylindrical arrangement of exchanging material 7002. For example, the exchanging material 7002 may be constructed using foam, corrugated materials, or a strip layer 7001 as described in relation to FIG. 7D.

The HME 7000 may include a clip 7005 configured to retain the cylindrical exchanging material 7002 within the HME frame in use. For example, the clip may be configured to engage with the inner surfaces of the HME frame 7003, to secure the exchanging material within or substantially within the HME frame 7003. As in previously described examples of the technology, the clip 7005 may be configured to releasably connect to the frame 7003 to allow the exchanging material to be removed and cleaned or replaced in use.

In the examples shown the clip 7005 comprises a central core 13002 around which the exchanging material 7002 is arranged in a coil, and lateral arms 13004 configured to engage with the inner walls of the frame 7003 in use. The lateral arms may be positioned at a first end of the central core, to define a substantially T-shaped clip 7005. In some examples of the technology the central core 1302 may be configured to attach to the exchanging material 7002, for example the exchanging material may be configured to attach to the central core using an adhesive, or other fastening mechanism, such as a clamp. One potential benefit of attaching the exchanging material to the central core can include a simplified assembly process.

The second end of the central core 13002 i.e., the end which is distal to the lateral arms 13004, may be configured to engage with a support member 13006 on the frame, to secure the central core to the frame, or otherwise aid with positioning the clip 7005 within the frame 7003. For example, the support member 13006 shown comprises an aperture 13008 configured to in-use receive a protrusion 13010 on the end of the clip 7005.

The lateral arms 13004 may be configured to engage with the exchanging material 7002 so as to prevent the exchanging material 7002 from moving outwardly of the frame 7003 once assembled. This arrangement may further assist with assembly of the HME 7000, as the clip 7005 and exchanging material 7002 may be pressed into the frame 7003 simultaneously.

The underside of the frame 7003 may comprise a plurality of protrusions 13012 which extend radially inwardly into the inside of the frame 7003. These protrusions 13012 may be provided proximate a single end of the frame 7003 so as to compress or otherwise shape the exchanging material 7002 within the HME 7000. For example, the protrusions 13012 may be provided as ribs, as illustrated in the present figures, or alternatively as a section of the frame 7003 which is narrower at one end, such as using a tapered frame construction, or using a thicker frame at one end.

These protrusions 13012 act on the exchanging material 7002 to compress a first end of the exchanging material such that the radially outer channels 7012 in the exchanging material are configured to provide a flow path in a direction D1, D2 which is not parallel with the longitudinal axis ‘L’ of the HME 7000. In other words, the radially outer channels are provided at an angle with respect to the longitudinal axis ‘L’ of the HME 7000. In some examples the angle may be between 5 and 25 degrees, such as between 5 and 15 degrees. Radially inner channels 7012, i.e., those positioned relatively close to central core 130002 may be oriented at less of an angle relative to the longitudinal axis of the HME 7000 than the radially outer channels.

It should be appreciated that the angles of the channels in the cylindrical HME 7000 of FIG. 13A to 13D may vary within the HME 7000. For example, the protrusions may be selected or provided to provide a channel pattern which gives a high level of CO2 washout or air flow diffusion within any given patient interface 3000.

FIG. 14 illustrates how the diffused HME 7000 constructions of an exemplary form of the present technology may be used to improve diffusion and CO2 washout within a patient interface. However, forms of the present technology are not limited to patient interfaces. For example, an HME in some forms of the present technology may be positioned inline between the patient interface and an air circuit 4170. For example, the angled channel constructions described herein may also be beneficial for any one or more of:
Directed expired air out through one or more vents 3400.
Diffusing the airflow prior to the airflow reaching the user's face, potentially improving comfort and use compliance.
Diffusing the expired airflow, to reduce vent 3400 noise, and partner disturbance.
Creating turbulent airflow within the patient interface to better washout CO2.

### 4.8 GLOSSARY

For the purposes of the present technology disclosure, in certain forms of the present technology, one or more of the following definitions may apply. In other forms of the present technology, alternative definitions may apply.

### 4.8.1 General

Air: In certain forms of the present technology, air may be taken to mean atmospheric air, and in other forms of the present technology air may be taken to mean some other combination of breathable gases, e.g., oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g., the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g., from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm 2 and is approximately 0.98 hectopascal (1 hectopascal=100 Pa=100 N/m²=1 millibar 0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 4.9 OTHER REMARKS

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means+/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1. A heat moisture exchanger for use in a respiratory therapy system, the heat moisture exchanger comprising:
a frame; and
an exchanging material situated at least partially within the frame,
wherein the exchanging material comprises a plurality of channels through which a flow of breathable gas passes in use,
wherein at least one of the channels is provided in a different orientation to at least one other channel.

2. The heat moisture exchanger of claim 1, wherein the plurality of channels comprises a first set of airflow channels and a second set of airflow channels, wherein the first set of airflow channels are provided in a first orientation and the second set of airflow channels are provided in a second orientation, wherein the first orientation is different from the second orientation.

3. The heat moisture exchanger of claim 1, wherein the exchanging material comprises a plurality of layers, wherein each of the plurality of layers comprises at least one channel.

4. The heat moisture exchanger of claim 3, wherein the first set of airflow channels is provided in a first layer of the plurality of layers, and the second set of airflow channels is provided in a second layer of the plurality of layers.

5. The heat moisture exchanger of claim 2, wherein the frame comprises a plurality of openings through which the flow of breathable gas flows in use, wherein the plurality of openings define a longitudinal axis of the heat moisture exchanger, and wherein the first orientation is substantially parallel to the longitudinal axis of the heat moisture exchanger.

6. The heat moisture exchanger of claim 2, wherein the frame comprises a plurality of openings through which the flow of breathable gas flows in use, wherein the plurality of openings define a longitudinal axis of the heat moisture exchanger, and wherein the first orientation is provided at an acute angle with respect to the longitudinal axis.

7. The heat moisture exchanger of claim 1, wherein the exchanging material comprises any one or more of: a corrugated material, foam, paper, textile or a composite material.

8. The heat moisture exchanger of claim 1, wherein the exchanging material comprises hygroscopic salts.

9. A patient interface for providing respiratory therapy treatment to a patient, the patient interface comprising:
a plenum chamber pressurisable to a therapeutic pressure of at least 4 cmH2O above ambient air pressure, wherein the plenum chamber includes at least one plenum chamber inlet port sized and is structured to receive a supply of breathable gas at the therapeutic pressure for breathing by a patient;
a seal-forming structure constructed and arranged to form a seal with a region of the patient's face surrounding an entrance to the patient's airways, wherein the seal-forming structure has a hole therein such that the supply of breathable gas is delivered to at least an entrance to the patient's nares, and wherein the seal-forming structure is constructed and arranged to maintain said therapeutic pressure in the plenum chamber throughout the patient's respiratory cycle in use;
a heat moisture exchanger comprising:
a frame; and
an exchanging material situated at least partially within the frame,
wherein the exchanging material comprises a plurality of channels through which a flow of breathable gas passes in use,
wherein at least one of the channels is provided in a different orientation to at least one other channel, and wherein the heat moisture exchanger is positioned in a flow path of the supply of breathable gas.

10. The patient interface of claim 9, further comprising a positioning and stabilising structure to provide a force to hold the seal-forming structure in a therapeutically effective position on the patient's head.

11. A system for providing a patient with a respiratory therapy treatment, comprising:
a patient interface comprised of:
a plenum chamber pressurisable to a therapeutic pressure of at least 4 cmH2O above ambient air pressure, wherein the plenum chamber includes at least one plenum chamber inlet port sized and is structured to receive a supply of breathable gas at the therapeutic pressure for breathing by a patient;
a seal-forming structure constructed and arranged to form a seal with a region of the patient's face surrounding an entrance to the patient's airways, wherein the seal-forming structure has a hole therein such that the supply of breathable gas is delivered to at least an entrance to the patient's nares, and wherein the seal-forming structure is constructed and arranged to maintain said therapeutic pressure in the plenum chamber throughout the patient's respiratory cycle in use; and
a heat moisture exchanger comprising:
a frame; and
an exchanging material situated at least partially within the frame, and
wherein the exchanging material comprises a plurality of channels through which a flow of breathable gas passes in use, wherein at least one of the channels is provided in a different orientation to at least one other channel, and wherein the heat moisture exchanger is positioned in a flow path of the supply of breathable gas;
a respiratory therapy apparatus configured to generate the supply of breathable gas in use, and
an air delivery circuit configured to facilitate transfer of the supply of breathable gas from the respiratory therapy apparatus to the patient interface.
